(19) United States
(12) Patent Application Publication
(10) Pub. No.: US 2024/0165183 A1
(43) Pub. Date: May 23, 2024

(54) METHODS AND COMPOSITIONS FOR MICROENCAPSULATED COMPOUNDS

(71) Applicant: OJAI ENERGETICS PBC, OJAI, CA (US)

(72) Inventor: William KLEIDON, Ojai, CA (US)

(73) Assignee: OJAI ENERGETICS PBC, OJAI, CA (US)

(21) Appl. No.: 18/420,673

(22) Filed: Jan. 23, 2024

Related U.S. Application Data
(63) Continuation of application No. PCT/US2022/038081, filed on Jul. 22, 2022.
(60) Provisional application No. 63/224,887, filed on Jul. 23, 2021.

(51) Int. Cl. A61K 36/185; A61K 9/107; A61K 9/48

(52) U.S. Cl. CPC A61K 36/185; A61K 9/107; A61K 9/4833

(57) ABSTRACT

The present disclosure provides a composition comprising a compound (e.g., a terpene compound and/or a cannabinoid compound). The composition may comprise an emulsion that comprises the encapsulated compound. In some cases, the emulsion may comprise a plurality of droplets, and a droplet of the plurality of droplets may comprise the encapsulated compound. In some cases, the emulsion may exhibit an effect or an experience to a user, which effect or experience is similar to that of a specific cannabis strain.

### CROSS-REFERENCE

This application is a continuation of PCT/US2022/038081, filed Jul. 22, 2022 which claims the benefit of U.S. Provisional Application Ser. No. 63/224,887, filed Jul. 23, 2021, which is entirely incorporated herein by reference.

### BACKGROUND

A cannabis plant can comprise a plurality of compounds, such as, for example, a plurality of cannabinoid compounds, a plurality of terpene compounds, etc. Different cannabis chemovars (or chemotypes) can comprise (e.g., naturally comprise) different amounts and/or ratios of such compounds. In some cases, a first ratio (e.g., a weight ratio) between one or more cannabinoid compounds and one or more terpene compounds naturally found in a first cannabis chemovar (e.g., a first strain of the cannabis plant) may be different than a second ratio (e.g., a weight ratio) between one or more cannabinoid compounds and one or more terpene compounds naturally found in a second cannabis chemovar (e.g., a second strain of the cannabis plant).

### SUMMARY

Different cannabis chemovars can induce different effects (e.g., different therapeutic effects) to a subject (e.g., when combusted or vaporized for consumption by the subject). There remains a substantial need for a composition (e.g., a manufactured emulsion composition, a non-natural emulsion composition) comprising a plurality of compounds, wherein a ratio (e.g., a weight ratio) of the plurality of compounds in the composition is similar to a ratio (e.g., a weight ratio) of the plurality of compounds of a cannabis chemovar or an extract thereof (e.g., that of a combusted or vaporized cannabis chemovar or an extract thereof). Having such similar ratio (or molecule profile) may induce a similar effect or experience to a subject that the cannabis chemovar or the extract naturally would.

Applicant recognizes that a tailored terpene profile may be generated for a given application of a cannabinoid. The effect of the cannabinoid may be modulated using the selected terpene profile. Applicant has recognized that various terpene combinations and concentrations, when administered with similar or identical cannabinoid compounds may result in different and distinct user experiences or effects of the cannabinoid compounds. Accordingly, Applicant has recognized that a terpene profile of a composition may affect, modulate, contribute to, or otherwise affect a user experience associated with administration of a composition comprising the terpene profile, particularly when the composition further comprises a cannabinoid.

In an aspect, provided in the present disclosure is a method of generating a cannabinoid formulation comprising (a) determining a terpene profile of a cannabis strain, wherein the terpene profile comprises at least a first terpene compound and a second terpene compound; (b) providing a first amount of the first terpene compound, a second amount of the second terpene compound, and a third amount of a cannabinoid compound to a reaction vessel, wherein in the reaction vessel a weight ratio of the first terpene compound to the second terpene compound is substantially equivalent to a weight ratio of the first terpene compound to the second terpene compound in the terpene profile determined in (a); (c) in the reaction vessel, mixing the first terpene compound, the second terpene compound, and the cannabinoid compound to generate an emulsion comprising the first terpene compound, the second terpene compound, and the cannabinoid compound.

In some embodiments, the emulsion comprises a plurality of droplets. An individual droplet of the plurality of droplets may have a size less than or equal to about 500 nanometers. The plurality of droplets may have a size distribution that is substantially homogenous. The droplet of the plurality of droplets may comprise the first terpene compound and the second terpene compound. The droplet may comprise the first terpene compound, the second terpene compound, and the cannabinoid compound.

In an aspect, provided is a composition comprising a terpene compound and a cannabinoid compound, wherein a weight ratio between the terpene compound and the cannabinoid compound in the composition (TC1) and a weight ratio between the terpene compound and the cannabinoid compound in a single cannabis strain (TC2) are different by no more than about 10%, wherein the composition is not a cannabis plant or a non-modified extract thereof.

In some embodiments, the composition further comprises an emulsion, wherein the emulsion comprises the terpene compound and the cannabinoid compound.

In some embodiments, the emulsion comprises a plurality of droplets, wherein a droplet of the plurality of droplets comprises the terpene compound and the cannabinoid compound.

In some embodiments, the emulsion comprises a plurality of droplets, wherein the terpene compound and the cannabinoid compound are in different droplets of the plurality of droplets.

In some embodiments, TC1 and TC2 are different by no more than about 10%.

In some embodiments, TC1 and TC2 are different by no more than about 5%.

In some embodiments, TC1 and TC2 are different by no more than about 1%.

In some embodiments, TC1 and TC2 are substantially the same.

In some embodiments, TC1 or TC2 is between about 100:1 and about 1:100.

In some embodiments, TC1 or TC2 is between about 50:1 and about 1:50.

In some embodiments, TC1 or TC2 is between about 30:1 and about 1:30.

In some embodiments, TC1 or TC2 is between about 1:10 and about 1:30.

In some embodiments, the composition does not comprise a full-spectrum cannabinoid profile of the cannabis plant.

In another aspect, provided is a composition comprising a plurality of terpene compounds comprising a terpene compound and an additional terpene compound that are different, wherein a weight ratio between the terpene compound and the additional terpene compound in the composition (TT1) and a weight ratio between the terpene compound and the additional terpene compound in a single cannabis strain (TT2) are different by no more than about 10%, and wherein the non-natural composition is substantially free of at least one type of terpene compound found in the cannabis plant, and wherein the composition is not a cannabis plant or a non-modified extract thereof.

In some embodiments, the composition further comprises an emulsion, wherein the emulsion comprises the plurality of terpene compounds.

In some embodiments, the emulsion comprises a plurality of droplets, wherein a droplet of the plurality of droplets comprises the terpene compound and the additional terpene compound.

In some embodiments, the emulsion comprises a plurality of droplets, wherein the terpene compound and the additional terpene compound are in different droplets of the plurality of droplets.

In some embodiments, TT1 and TT2 are different by no more than about 10%.

In some embodiments, TT1 and TT2 are different by no more than about 5%.

In some embodiments, TT1 and TT2 are different by no more than about 1%.

In some embodiments, TT1 and TT2 are substantially the same.

In some embodiments, TT1 or TT2 is between about 100:1 and about 1:100.

In some embodiments, TT1 or TT2 is between about 50:1 and about 1:50.

In some embodiments, TT1 or TT2 is between about 30:1 and about 1:30.

In some embodiments, TT1 or TT2 is between about 10:1 and about 1:10.

In some embodiments, the composition is substantially free of a plurality of types of terpene found in the cannabis plant.

In some embodiments, the composition does not comprise a full-spectrum terpene profile of the cannabis plant.

In some embodiments, the composition further comprises a cannabinoid compound.

In some embodiments for any of the compositions described herein, the single cannabis strain is selected from the group consisting of 9 Pound Hammer, Afghani, Afgoo, Berry White, Blueberry, Bubba Kush, G13, Granddaddy Purple, Grape Ape, Herijuana, Hindu Kush, Ingrid, Kosher Kush, Lavender, Master Kush, Northern Lights, Obama Kush, Pez, Plushberry, Presidential OG, Purple Urkle, Willy's Wonder, Zkittlez, Acdc, Ak-47, Banana OG, Bluc Dream, Cannatonic, Chemdawg, Chernobyl, Cherry Pie, Cinderella 99, Dancehall, Double Dream, Dutch Treat, Ewok, Fruity Pebbles, Gelato, Golden Goat, Headband, Jeanguy, Jellybean, Juicy Fruit, Larry OG, Lemonder, Lodi Dodi, Mango Kush, Mendocino Purps, Middlefork, OG Kush, Permafrost, Pineapple Chunk, Pineapple Express, Pin Kush, Rascal OG, Sage, Sfv OG, Shiatsu Kush, Skunk No. 1, Snoop's Dream, Snowcap, Sour OG, Sour Tsunami, Space Queen, Sunset Sherbet, Tahoe OG, Tangerine Dream, Trainwreck, Uk Cheese, White Fire OG, White Widow, X3-X13, Acapulco Gold, Allen Wrench, Amnesia, Bay 11, Chocolope, Cinex, Dirty Girl, Durban Poison, Ghost Train Haze, Grapefuirt, Green Crack, Harlequin, Island Sweet Skunk, Jack Herer, Kali Mist, Lamb's Bread, Laghing Buddha, Maui Wowie, Panana Red, Purple Haze, Red Headed Stranger, Schrom, Sour Diesel, Strawberry Cough, Super Lemon Haze, Super Silver Haze, Tangie, Acapulco Gold, African, Cambodian red, Colombian, Hawaiian, Jamaican gold, Mexican red, Panama red, Thai stick, Amnesia Haze, Blueberry Diesel, Blue Goo, Bruce Banner, Bubblegum, Charlotte's Web, Critical Mass, Girl Scout Cookies, Gorilla Gluc (GG4), Haze, Maui Waui, Platinum OG, Paonia Purple, Willie Nelson, Tochigishiro, and Feral cannabis.

In some embodiments for any of the compositions described herein, the terpene compound or the additional terpene compound is selected from the group consisting of myrcene, limonene, linalool, trans-ocimene, cis-ocimene, alpha-pinene, beta-pinene, alpha-humulene (alpha-caryophyllenc), beta-caryophyllene, delta-3-carene, trans-gamma-bisabolene, cis-gamma-bisabolene, trans-alpha-farnesene, cis-beta-farnesene, beta-fenchol, beta-phellandrene, guajol, alpha-gualenc, alpha-cudesmol, beta-cudesmol, gamma-eudesmol, terpinolene, alpha-selinene, beta-selinene, alpha-terpineol, fenchone, camphene, cis-sabinene hydrate, alpha-trans-bergamotene, alpha-cis-bergamotene, borncol, gamma-curcumene, alpha-thujene, cpi-alpha-bisabolol, ipsdienol, alpha-ylangene, beta-clemenc, gamma-muurolenc, alpha-cadinenc, alpha-longipinene, and caryophyllene oxide.

In some embodiments for any of the compositions described herein, the cannabinoid compound is selected from the group consisting of cannabigerol-type (CBG), cannabigerolic acid (CBGA), cannabigerolic acid monomethylether (CBGAM), cannabigerol monomethyl ether (CBGM), cannabichromene-type (CBC), cannabichromanon (CBCN), cannabichromenic acid (CBCA), cannabichromevarin-type (CBCV), cannabichromevarinic acid (CBCVA), cannabidiol-type (CBD), tetrahydrocannabinol-type (THC), iso-tetrahydrocannabinol-type (iso-THC), cannabinol-type (CBN), cannabinolic acid (CBNA), cannabinol methylether (CBNM), cannabinol-C4 (CBN-C4), cannabinol-C2 (CBN-C2), cannabiorcol (CBN-C1), cannabinodiol (CBND), cannabielsoin-type (CBE), cannabielsoic acid A (CBEA-A), cannabielsoic acid B (CBEA-B), cannabicyclol-type (CBL), cannabicyclolic acid (CBLA), cannabicyclovarin (CBLV), cannabicitran-type (CBT), cannabitriol, cannabitriolvarin (CBTV), ethoxy-cannabitiolvarin (CBTVE), cannabivarin-type (CBV), cannabinodivarin (CBVD), tetrahydrocannabivarin-type (THCV), cannabidivarin-type (CBDV), cannabigerovarin-type (CBGV), cannabigerovarinic acid (CBGVA), cannabifuran (CBF), dehydrocannabifuran (DCBF), and cannabiripsol (CBR) cannabinoids.

In some embodiments for any of the compositions described herein, the cannabinoid compound comprises CBD.

In some embodiments for any of the compositions described herein, the cannabinoid compound comprises THC.

In some embodiments for any of the compositions described herein, the cannabinoid compound comprises a plurality of cannabinoid compounds.

In some embodiments for any of the compositions described herein, the composition comprises a flavonoid.

In another aspect, provided is a method for forming a composition comprising a terpene compound and a cannabinoid compound, the method comprising generating a mixture comprising the terpene compound and the cannabinoid compound, wherein a weight ratio between the terpene compound and the cannabinoid compound in the mixture and a weight ratio between the terpene compound and the cannabinoid compound in a single cannabis strain (TC2) are different by no more than about 10%, and wherein the composition is not a cannabis plant or a non-modified extract thereof.

In another aspect, provided is a method for forming a composition comprising a plurality of terpene compounds comprising a terpene compound and an additional terpene compound that are different, the method comprising generating a mixture comprising the plurality of terpene compounds, wherein a weight ratio between the terpene compound and the additional terpene compound in the non-natural composition (TT1) and a weight ratio between the terpene compound and the additional terpene compound in a single cannabis strain (TT2) are different by no more than about 10%, wherein the non-natural composition is substantially frec of at least one type of terpene compound found in the cannabis plant, and wherein the composition is not a cannabis plant or a non-modified extract thereof.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIGS. 1A-1C show different compositions, each composition comprising a terpene compound and a cannabinoid compound;

FIG. 1D shows a cannabis plant composition or an extract thereof, comprising a terpene compound and a cannabinoid compound;

FIGS. 2A-2C show different compositions, each composition comprising a terpene compound and an additional compound; and

FIG. 2D shows a cannabis plant composition or an extract thereof, comprising a terpene compound and an additional compound.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term “about,” as used herein, generally refers to an acceptable error range for the particular value as determined by one of ordinary skill in the art, which may depend in part on how the value is measured or determined. For example, “about” can mean within 1 or more than 1 standard deviation. Alternatively, “about” can mean a range of up to 20%, up to 10%, up to 5%, or up to 1% of a given value. Alternatively, particularly with respect to biological systems or processes, the term can mean within an order of magnitude, within 5-fold, and within 2-fold, of a value.

The term “subject,” as used herein, generally refers to an animal. The subject may have or be suspected of having a disease or ailment. A subject may be a mammal. Non-limiting examples of mammals include humans and animals, such as mice, monkeys, dogs and cats, including transgenic and non-transgenic mice. The methods described herein can be useful in both human therapeutics, pre-clinical, and veterinary applications. The subject may be a mammal. The subject may be human. Other mammals include, but are not limited to, apes, chimpanzees, orangutans, monkeys; domesticated animals (pets) such as dogs, cats, guinea pigs, hamsters, mice, rats, rabbits, and ferrets; domesticated farm animals such as cows, buffalo, bison, horses, donkey, swine, sheep, and goats; or exotic animals typically found in zoos, such as bear, lions, tigers, panthers, elephants, hippopotamus, rhinoceros, giraffes, antelopes, sloth, gazelles, zebras, wildebeests, prairie dogs, koala bears, kangaroo, pandas, giant pandas, hyena, seals, sea lions, and elephant seals.

The term “administer,” as used herein, generally refers to providing a composition to a subject via a route of administration, including but not limited to intravenous, intraarterial, oral, parenteral, buccal, topical, transdermal, rectal, intramuscular, subcutaneous, intraosseous, transmucosal, or intraperitoneal routes of administration. A composition may be administered via a suppository, such as a vaginal or anal suppository. Oral routes of administration may be used. A unit dose may be administered via inhalation. Examples of the administration method may include, but are not limited to, intradermal, subcutaneous, intramuscular, intravenous, intraosseous, intraperitoneal, intrathecal, epidural, intracardiac, intraarticular, intracavernous, intravitreal, transdermal, and/or oral (e.g., buccal, sublabial, and/or sublingual). Examples of dosage forms for the oral administration can include, but are not limited to, a capsule, dragee, pill, tablet, gel, liquid, slurry, suspension, syrup, and powder. Examples transdermal administration can include, but are not limited to, a transdermal patch, a topical liquid or gel, immersion therapy (e.g., subjecting an individual in a float tank).

The term “effective amount” or “therapeutically effective amount,” as used herein, generally refers to an amount of a compound described herein that is sufficient to affect an intended, predetermined or prescribed application, including but not limited to, disease or condition treatment. The therapeutically effective amount can vary depending upon the application (e.g., in vitro or in vivo), or the subject and disease condition being treated, e.g., the weight and age of the subject, the severity of the disease condition and the manner of administration. The term also may apply to a dose that induces a particular response in target cells, e.g., reduction of proliferation or down regulation of activity of a target protein. The specific dose may vary depending on the particular compounds chosen, the dosing regimen to be followed, whether it is administered in combination with other compounds, timing of administration, the tissue to which it is administered, and the physical delivery system in which it is carried.

The term “isolated,” as used herein, generally refers to a preparation of a substance devoid of at least some of the other components that can also be present where the substance or a similar substance naturally occurs or is initially obtained from. Thus, for example, an isolated substance can be prepared by using a purification technique to enrich it from a source mixture. Enrichment can be measured on an absolute basis, such as weight per volume of solution, or it can be measured in relation to a second, potentially interfering substance present in the source mixture. Increasing enrichment may be used. A substance can also be provided in an isolated state by a process of artificial assembly, such as by chemical synthesis.

The term “substantially free,” as used herein, generally refers to a composition that has less than about 25% (e.g., by weight), less than about 15%, less than about 10%, less than about 5%, less than about 1%, less than about 0.5%, less than 0.1% or even less of a specified component. Such composition may not have a detectable amount of such specified component. For example a composition that is substantially free of a weak acid (e.g., an acid with a pKa of at most about 10) can have less than about 1% of the weak acid. The percentage can be determined as a percent of the total composition or a percent of a subset of the composition. For example, a composition that is substantially free of a weak acid can have less than 1% of the weak acid as a percent of the total composition, or as a percent of the acids in the composition. The percentages can be mass, molar, or volume percentages. The presence or concentration of such component may be determined spectroscopically, such as chromatography or nuclear magnetic resonance.

The term “cannabis plant,” as used herein, generally refers to a plant that is part of a genus of a flowering plant in the family Cannabaceae, and may include three species or subspecies: sativa, indica, and ruderalis. A cannabis plant may comprise a number of different parts, including a node, a plant stem, a fan leaf, and a flower. The flower of a cannabis plant may be a male or a female flower. The female flower may comprise a flower, a pistil, a cola, a trichome, and a calyx.

The term “chemovar” or “chemotype,” as used interchangeably herein, generally refers to a chemically distinct entity in a plant, such as a cannabis plant. A cannabis chemovar can comprise a unique composition, such as one or more terpene compounds and one or more cannabinoid compounds. Consumption (e.g., via combustion or vaporization) of a first cannabis chemovar can induce a different effect than consumption (e.g., via combustion or vaporization) of a second cannabis chemovar. For example, an individual can have different experiences by consuming (e.g., via combustion or vaporization) different cannabis chemovars.

A cannabis chemovar may be a specific cannabis strain may be indica, sativa, or hybrid. Non-limiting examples of a cannabis strain can include 9 Pound Hammer, Afghani, Afgoo, Berry White, Blueberry, Bubba Kush, G13, Granddaddy Purple, Grape Ape, Herijuana, Hindu Kush, Ingrid, Kosher Kush, Lavender, Master Kush, Northern Lights, Obama Kush, Pez, Plushberry, Presidential OG, Purple Urkle, Willy's Wonder, Zkittlez, Acdc, Ak-47, Banana OG, Blue Dream, Cannatonic, Chemdawg, Chernobyl, Cherry Pie, Cinderella 99, Dancehall, Double Dream, Dutch Treat, Ewok, Fruity Pebbles, Gelato, Golden Goat, Headband, Jeanguy, Jellybean, Juicy Fruit, Larry OG, Lemonder, Lodi Dodi, Mango Kush, Mendocino Purps, Middlefork, OG Kush, Permafrost, Pineapple Chunk, Pineapple Express, Pin Kush, Rascal OG, Sage, Sfv OG, Shiatsu Kush, Skunk No. 1, Snoop's Dream, Snowcap, Sour OG, Sour Tsunami, Space Queen, Sunset Sherbet, Tahoe OG, Tangerine Dream, Trainwreck, Uk Cheese, White Fire OG, White Widow, X3-X13, Acapulco Gold, Allen Wrench, Amnesia, Bay 11, Chocolope, Cinex, Dirty Girl, Durban Poison, Ghost Train Haze, Grapefuirt, Green Crack, Harlequin, Island Sweet Skunk, Jack Herer, Kali Mist, Lamb's Bread, Laghing Buddha, Maui Wowie, Panana Red, Purple Haze, Red Headed Stranger, Schrom, Sour Diesel, Strawberry Cough, Super Lemon Haze, Super Silver Haze, Tangie, Acapulco Gold, African, Cambodian red, Colombian, Hawaiian, Jamaican gold, Mexican red, Panama red, Thai stick, Amnesia Haze, Blueberry Diesel, Bluc Goo, Bruce Banner, Bubblegum, Charlotte's Web, Critical Mass, Girl Scout Cookies, Gorilla Gluc (GG4), Haze, Maui Waui, Platinum OG, Paonia Purple, Willie Nelson, Tochigishiro, and Feral cannabis.

The term “cannabinoid,” as used herein, generally refers to a cannabinoid compound that has been isolated or identified in a cannabis plant. A cannabinoid compound may act on a cannabinoid receptor in a cell. The cannabinoid may alter physiological processes, including altering neurotransmitter release in the brain, appetite, pain-sensation, mood, and memory. A cannabinoid compound may have a C₂₁ terpenophenolic core.

A cannabinoid compound as disclosed herein in may comprise one or more carboxylated cannabinoid compounds. Alternatively or in addition to, the cannabinoid compound may comprise one or more decarboxylated cannabinoid compounds. The term “carboxylated cannabinoid,” as used herein, generally refers to a compound that has been isolated or identified in a cannabis plant and possesses a carboxylic acid moiety (i.e. —COOH). A carboxylated cannabinoid may be tetrahydrocannabinolic acid. The term “decarboxylated cannabinoid,” as used herein, generally refers to a cannabinoid compound that previously possessed a carboxylic acid moiety (e.g. a carboxylated cannabinoid), and underwent a chemical reaction so to no longer possesses the carboxylic acid moiety. A decarboxylated cannabinoid may be a natural compound and may be present in a cannabis plant. A decarboxylated cannabinoid may be synthesized or produced via synthetic methods. A decarboxylated cannabinoid may be 49 tetrahydrocannabinol.

The term “terpenoid,” as used herein, generally refers to an organic compound that is composed of isoprene units, wherein each isoprene unit has the formula C₅H₈. The isoprene units may be connected via covalent bonds. Terpenoids may have the formula (C₅H₈)ₙ, wherein n is an integer of 1 or more, such as 2, 3, 4, 5, or more. A terpenoid may be a monoterpenoid (C₁₀ skeleton), sesquiterpenoid (C₁₅ skeleton), diterpenoid (C₂₀ skeleton), or treterpenoid (C₃₀ skeleton). Terpenoids may possess beneficial effects on a subject, and may be used as a dietary supplement or nutritional supplement.

In some embodiments, cannabinoid compounds can be divided into ten subclasses. Subclasses of cannabinoid compounds include the cannabigerol class, cannabichromene class, cannabidiol class, delta-9-tetrahydrocannabinol class, delta-8-tetrahydrocannabinol class, cannabicyclol class, cannabielsoin class, cannabinol and cannabinodiol class, cannabitriol class, and a miscellaneous cannabinoids class.

Non-limiting examples of cannabinoid compounds in the cannabigerol class include cannabigerolic acid (CBGA), cannabigerolic acid monomethylether (CBGAM), cannabigerol (CBG), cannabigerol monomethylether (CBGM), cannabigerovarinic acid (CBGVA), and cannabigerovarin (CBGV).

Non-limiting examples of cannabinoid compounds in the cannabichromene class include cannabichromenic acid (CBCA), cannabichromene (CBC), cannabichromevarinic acid (CBCVA), and cannabichromevarin (CBCV).

Non-limiting examples of cannabinoid compounds in the cannabidiol class include cannabidiolic acid (CBDA), cannabidiol (CBD), cannabidiol monomethylether (CBDM), cannabidiol-C₄ (CBD-C₄), cannabidivarinic acid (CBDVA), cannabidivarin (CBDV), and cannabidiorcol (CBD-C₁).

Non-limiting examples of cannabinoid compounds in the delta-9-tetrahydrocannabinol class include delta-9-tetrahydrocannabinolic acid A (THCA-A), delta-9-tetrahydrocannabinolic acid B (THCA-B), delta-9-tetrahydrocannabinol (THC), delta-9-tetrahydrocannabinolic acid-C₄ (THCA-C₄), delta-9-tetrahydrocannabinol-C₄ (THC-C₄), dena-9-tetrahydrocannabivarinic acid (THCVA), delta-9-tetrahydrocannabivarin (THCV), delta-9-tetrahydrocannabiorcolic acid (THCA-C₁), delta-9-tetrahydrocannabiorcol (THC-C₁), and delta-7-cis-iso-tetrahydrocannabivarin.

Non-limiting examples of cannabinoid compounds in the delta-8-tetrahydrocannabinol class include delta-8-tetrahydrocannabinolic acid (48-THCA), and delta-8-tetrahydrocannabinol (48-THC).

Non-limiting examples of cannabinoid compounds in the cannabicyclol class include cannabicyclolic acid (CBLA), cannabicyclol (CBL), and cannabicyclovarin (CBLV).

Non-limiting examples of cannabinoid compounds in the cannabielsoin class include cannabielsoic acid A (CBEA-A), cannabielsoic acid B (CBEA-B), and cannabiclsoin (CBE).

Non-limiting examples of cannabinoid compounds in the cannabinol and cannabinodiol class include cannabinolic acid (CBNA), cannabinol (CBN), cannabinol methylether (CBNM), cannabinol-C₄ (CBN-C₄), cannabivarin (CBV), cannabinol-C₂ (CBN-C₂), cannabiorcol (CBN-C₁), cannabinodiol (CBND), and cannabinodivarin (CBVD).

Non-limiting examples of cannabinoid compounds in the cannabitriol class include cannabitriol (CBT), 10-ethoxy-9-hydroxy-delta-6a-tetrahydrocannabinol, 8,9-dihydroxy-delta-6a-tetrahydrocannabinol, cannabitriolvarin (CBTV), and ethoxy-cannabitriolvarin (CBTVE).

Non-limiting examples of cannabinoid compounds in the miscellaneous cannabinoids class, include dehydrocannabifuran (DCBF), cannabifuran (CBF), cannabichromanon (CBCN), cannabicitran (CBT), 10-oxo-delta-6a-tetrahydrocannabinol (OTHC), delta-9-cis-tetrahydrocannabinol (cis-THC), 3,4,5,6-tetrahydro-7-hydroxy-alpha-alpha-2-trimethyl-9-n-propyl-2,6-methano-2H-1-benzoxocin-5-methanol (OH-iso-HHCV), cannabiripsol (CBR), and trihydroxy-delta-9-tetrahydrocannabinol (triOH-THC).

Terpenoid compounds that have been isolated from the essential oil of a cannabis plant may include myrcene, limonene, linalool, trans-ocimene, beta-pinene, alpha-pinene, beta-caryophyllene, delta-3-carene, trans-gamma-bisabolene, trans-alpha-farnesene, beta-fenchol, beta-phellandrene, alpha-humulene (alpha-caryophyllenc), guajol, alpha-guaiene, alpha-cudesmol, terpinolene, alpha-selinene, alpha-terpineol, fenchone, camphene, cis-sabinene hydrate, cis-ocimene, beta-cudesmol, beta-selinene, alpha-trans-bergamotene, gamma-cudesmol, borncol, cis-beta-farnesene, gamma-curcumene, cis-gamma-bisabolene, alpha-thujene, cpi-alpha-bisabolol, ipsdienol, alpha-ylangene, beta-elemene, alpha-cis-bergamotene, gamma-muurolene, alpha-cadinene, alpha-longipinene, and caryophyllene oxide.

Nitrogen-containing compounds have been isolated from a cannabis plant. Spermidine-type alkaloids that have been isolated from Cannabis sativa may include cannabisativine and anhydrocannabisativine. Other nitrogen-containing compounds that have been isolated from a cannabis plant include, but is not limited to, n-trans-feruloyityramine, n-p-coumaroyltyramine, n-trans-caffeoyltyramine, grossamide, cannabisin-A, cannabisin-B, cannabisin-C, and cannabisin-D.

Flavonoids are compounds that may be plant or fungus secondary metabolites. Generally, flavonoids have a Cis skeleton. Flavonoids have been identified in a cannabis plant, including apigenin, luteolin, kaempferol, quercetin, orientin, vitexin, cannflavin A, and cannflavin B.

Additional compounds that have been isolated from a cannabis plant include unsaturated fatty acids and noncannabinoid phenols, including, but not limited to, linoleic acid, alpha-linolenic acid, oleic acid, cannabispiran, isocannabispiran, cannabistilbene-I, cannabistilbene-II, cannithrene-1, and cannithrene-2.

### Compositions and Methods Thereof

The term “non-natural composition” as used herein may generally refer to a composition that may not be a cannabis plant for a non-modified extract thereof. A non-natural composition may comprise synthetic compounds. Alternatively or in addition to, a non-natural composition may comprise at least a portion of an extract of a cannabis plant and one or more additional compounds. A non-natural composition may be a manufactured composition (e.g., a manufactured emulsion composition) comprising synthetic compounds and/or an extract of a cannabis plant.

In some embodiments, administration (e.g., oral, intravenous, topical etc.) of a cannabis plant-derived composition or a synthetic composition thereof (e.g., edibles or topicals with fat soluble extracts) may not induce the same effect (e.g., therapeutic effect) or experience to an individual as that which the individual would normally experience via combustion or vaporization of such cannabis plant or an extract thereof. In some cases, consumption via combustion or vaporization of a cannabis chemovar may have a unique or nuanced effect on the individual, which may not be recapitulated by administration of an extract of the same cannabis chemovar via routes that are not combustion or vaporization. For examples, terpenes may be degraded (e.g., heavily degraded) via first pass digestion upon such administration that is not combustion or vaporization. In other examples, topical administration of compounds may not lead to systemic delivery of such compounds, and such compounds may primarily stay in dermal layers.

In some embodiments, the compositions disclosed herein are prepared by adding one or more terpene compounds in a ratio (e.g., a weight ratio) that is substantially the same as that in a cannabis plant or an extract thereof. The compositions disclosed herein may comprise emulsions, such as water soluble droplets (e.g., single layer droplets such as colloids). The compositions disclosed herein may elicit a unique user experience that may be substantially the same as that by the particular cannabis plant strain (e.g., experience via combustion or vaporization of the particular cannabis plant strain). The compositions disclosed herein may be administrated via, for example, ingestion, sublingual administration, buccal administration, topical administration, suppository, or intravenous administration.

In some embodiments, the onset time and/or offset time of the effect/experience exerted on the user upon administration of the compositions disclosed herein (e.g., monolayer encapsulation compositions) may be similar (e.g., substantially the same) as that exerted on the user upon combustion or vaporization of a respective cannabis chemotype or an extract thereof. In some embodiments, the compositions disclosed herein may not comprise liposomes. Without wishing to be found by theory, liposomal compositions may be characterized by having a faster onset (e.g., that of an experience in an individual) than non-liposomal compositions (e.g., fat soluble compositions). Without wishing to be bound by theory, liposomal compositions may be characterized by having a slower onset degree (e.g., that of an experience in an individual) and reduced strain-specific effect than non-liposomal compositions (e.g., monolayer encapsulation compositions).

In some embodiments, the compositions disclosed herein may comprise an emulsion that comprises synthetic compounds. In some embodiments, the compositions disclosed herein may comprise an emulsion that comprises an extract of a cannabis chemovar (e.g., a rosin derived or obtained from a single cannabis strain). In some embodiments, the compositions disclosed herein may comprise an emulsion that comprises one or more synthetic compounds and an extract of a cannabis chemovar.

In some embodiments, flavor kits for beverages or edibles that contain terpene and/or cannabinoid profiles that are from or match specific cannabinoid chemovars can be made to be put into various product formats (e.g., solid, liquid, gel, powder, paste, etc.) with final format containing encapsulated (e.g., monolayer encapsulated) compounds, such as cannabinoids and/or terpenes. One or more additional flavor compounds can be added to such kits, e.g., a margarita flavor.

In some embodiments, the compositions (e.g., non-natural compositions) disclosed herein can exert a similar effect (e.g., a therapeutic effect or an experience) in a subject that is exerted to the subject upon consumption (e.g., via combustion or vaporization) of a specific cannabis chemovar or an extract thereof.

In some embodiments, the compositions (e.g., non-natural compositions) disclosed herein can match a ratio (e.g., a weight ratio) of a first compound (e.g., a cannabinoid compound) and a second compound (e.g., a terpene compound) that is found naturally in a specific cannabis chemovar or an extract thereof.

In some embodiments, the compositions disclosed herein may comprise a plurality of compounds (e.g., a terpene compound and a cannabinoid compound, a plurality of different terpene compounds, a plurality of different cannabinoid compounds, etc.). The plurality of compounds may be encapsulated (e.g., via an emulsion) in the compositions. Alternatively, only a portion of the plurality of compounds may be encapsulated, while the other portion of the plurality of compounds may not be encapsulated. For example, a composition may comprise (i) encapsulated cannabinoid compounds (e.g., cannabinoid compounds in monolayer droplets) and (ii) terpene compounds that are either non-encapsulated or encapsulated in particles that do not comprise the cannabinoid compounds.

In some embodiments, by administering the compositions disclosed herein to a subject (e.g., via oral consumption, topical administration, intravenous administration, etc.), the subject may obtain an effect (e.g., a therapeutic effect) or an experience that is substantially similar to that from a specific cannabis chemovar (e.g., Sour Diesel, OG Kush, etc.), but without having to combust or vaporize the specific cannabis chemovar.

In some embodiments, an onset of an effect or experience induced to the subject (e.g., duration of time it takes for the effect or the experience to come to prominence) upon administering the compositions disclosed herein to a subject (e.g., via oral consumption, topical administration, intravenous administration, etc.) (ON1) and an onset of the effect or experience induced to the subject upon consumption (e.g., via combustion or vaporization) of a cannabis strain (e.g., a single cannabis strain) (ON2) may be different from each other by no more than about 20%, 15%, 10%, 9%, 8%, 7%, 6%, 5%, 4%, 3%, 2%, 1.5%, 1%, 0.5%, 0.1%, 0.05%, 0.01%, or less. In some cases, ON1 and ON2 may be substantially the same. In some cases, ON1 or ON2 may be at least about 1 minute, 2 minutes, 3 minutes, 4 minutes, 5 minutes, 10 minutes, 15 minutes, 20 minutes, 25 minutes, 30 minutes, 40 minutes, 50 minutes, 60 minutes, 2 hours, 3 hours, 6 hours, 9 hours, 12 hours, 24 hours, or more. In some cases, ON1 or ON2 may be at most about 24 hours, 12 hours, 9 hours, 6 hours, 3 hours, 2 hours, 60 minutes, 50 minutes, 40 minutes, 30 minutes, 25 minutes, 20 minutes, 15 minutes, 10 minutes, 5 minutes, 4 minutes, 3 minutes, 2 minutes, 1 minute, or less.

In some embodiments, a duration of an effect or experience induced to the subject (e.g., duration of time it takes for the effect or the experience to come to prominence) upon administering the compositions disclosed herein to a subject (e.g., via oral consumption, topical administration, intravenous administration, etc.) (DU1) and a duration of the effect or experience induced to the subject upon consumption (e.g., via combustion or vaporization) of a cannabis strain (e.g., a single cannabis strain) (DU2) may be different from each other by no more than about 20%, 15%, 10%, 9%, 8%, 7%, 6%, 5%, 4%, 3%, 2%, 1.5%, 1%, 0.5%, 0.1%, 0.05%, 0.01%, or less. In some cases, DU1 and DU2 may be substantially the same. In some cases, DU1 or DU2 may be at least about 5 minutes, 10 minutes, 15 minutes, 20 minutes, 25 minutes, 30 minutes, 40 minutes, 50 minutes, 60 minutes, 2 hours, 3 hours, 6 hours, 9 hours, 12 hours, 24 hours, or more. In some cases, ON1 or ON2 may be at most about 24 hours, 12 hours, 9 hours, 6 hours, 3 hours, 2 hours, 60 minutes, 50 minutes, 40 minutes, 30 minutes, 25 minutes, 20 minutes, 15 minutes, 10 minutes, 5 minutes, or less.

In some embodiments, an offset (or terminal plasma half-life) of the composition as disclosed herein upon its administration to a subject (e.g., via oral consumption, topical administration, intravenous administration, etc.) (OFF1) and an offset of a cannabis strain (e.g., a single cannabis strain) (OFF2) upon its consumption (e.g., via combustion or vaporization) may be different from each other by no more than about 20%, 15%, 10%, 9%, 8%, 7%, 6%, 5%, 4%, 3%, 2%, 1.5%, 1%, 0.5%, 0.1%, 0.05%, 0.01%, or less. In some cases, a terminal plasma half-life may be the time required to divide the plasma concentration of one or more compounds of interest by two after reaching pseudo-equilibrium. In some cases, OFF1 and OFF2 may be substantially the same. In some cases, OFF1 or OFF2 may be at least about 1 minute, 2 minutes, 3 minutes, 4 minutes, 5 minutes, 10 minutes, 15 minutes, 20 minutes, 25 minutes, 30 minutes, 40 minutes, 50 minutes, 60 minutes, 2 hours, 3 hours, 6 hours, 9 hours, 12 hours, 24 hours, or more. In some cases, OFF1 or OFF2 may be at most about 24 hours, 12 hours, 9 hours, 6 hours, 3 hours, 2 hours, 60 minutes, 50 minutes, 40 minutes, 30 minutes, 25 minutes, 20 minutes, 15 minutes, 10 minutes, 5 minutes, 4 minutes, 3 minutes, 2 minutes, 1 minute, or less.

Compositions as disclosed herein may be non-natural compositions. A non-natural composition may comprise at least one terpene compound (e.g., at least 1, 2, 3, 4, 5, 6, 7, 8, 9, 10, 15, 20, or more different types of terpene compounds) and at least one cannabinoid compound (e.g., at least 1, 2, 3, 4, 5, 6, 7, 8, 9, 10, 15, 20, or more different types of cannabinoid compounds). The at least one terpene compound and the at least one cannabinoid compound may be from the same source (e.g., both synthetically produced, both from a same cannabis strain) or may be from different sources (e.g., one synthetically produced while the other extracted from a cannabis strain). In some cases, a weight ratio between the at least one terpene compound and the at least one cannabinoid compound in such non-natural composition (TC1) and a weight ratio between the at least one terpene compound and the at least one cannabinoid compound in a cannabis strain (e.g., a single cannabis strain) (TC2) may be different from cach other by no more than about 20%, 15%, 10%, 9%, 8%, 7%, 6%, 5%, 4%, 3%, 2%, 1.5%, 1%, 0.5%, 0.1%, 0.05%, 0.01%, or less. In some cases, TC1 and TC2 may be substantially the same.

In some cases, TC1 or TC2 may be between about 500:1 and about 1:500, about 200:1 and about 1:200, about 100:1 and about 1:100, about 80:1 and about 1:80, about 60:1 and about 1:60, about 50:1 and about 1:50, about 40:1 and about 1:40, about 30:1 and about 1:30, about 20:1 and about 1:20, about 10:1 and about 1:10, about 5:1 and about 1:5, or about 2:1 and about 1:2. In some cases, TC1 or TC2 may be about 500:1, 200:1, 100:1, about 90:1, about 80:1, about 70:1, about 60:1, about 50:1, about 40:1, about 30:1, about 20:1, about 15:1, about 10:1, about 5:1, about 2:1, about 1:1, about 1:2, about 1:5, about 1:10, about 1:15, about 1:20, about 1:30, about 1:40, about 1:50, about 1:60, about 1:70, about 1:80, about 1:90, about 1:100, about 1:200, or about 1:500.

In some cases, the non-natural compositions as disclosed herein does not comprise a full-spectrum cannabinoid profile of a cannabis plant. For example, as compared to a control cannabis plant, the non-natural composition may lack at least one type of cannabinoid (e.g., at least 1, 2, 3, 4, 5, or more types of cannabinoid compounds normally found in the control cannabis plant may be missing in the non-natural composition). Alternatively, substantially all types of cannabinoid compounds naturally present in the control cannabis plant may be found in the non-natural composition.

Compositions as disclosed herein may be non-natural compositions. A non-natural composition may comprise a plurality of terpene compound types (e.g., at least 1, 2, 3, 4, 5, 6, 7, 8, 9, 10, 15, 20, or more different types of terpene compounds), such as a first terpene compound type and a second terpene compound type that are different. The first terpene compound and the second terpene compound may be from the same source (e.g., both synthetically produced, both from a same cannabis strain) or may be from different sources (e.g., one synthetically produced while the other extracted from a cannabis strain). In some cases, a weight ratio between the first terpene compound and the second terpene compound in such non-natural composition (TT1) and a weight ratio between the first terpene compound and the second terpene compound in a cannabis strain (e.g., a single cannabis strain) (TT2) may be different from each other by no more than about 20%, 15%, 10%, 9%, 8%, 7%, 6%, 5%, 4%, 3%, 2%, 1.5%, 1%, 0.5%, 0.1%, 0.05%, 0.01%, or less. In some cases, TT1 and TT2 may be substantially the same.

In some cases, TT1 or TT2 may be between about 500:1 and about 1:500, about 200:1 and about 1:200, about 100:1 and about 1:100, about 80:1 and about 1:80, about 60:1 and about 1:60, about 50:1 and about 1:50, about 40:1 and about 1:40, about 30:1 and about 1:30, about 20:1 and about 1:20, about 10:1 and about 1:10, about 5:1 and about 1:5, or about 2:1 and about 1:2. In some cases, TT1 or TT2 may be about 500:1, 200:1, 100:1, about 90:1, about 80:1, about 70:1, about 60:1, about 50:1, about 40:1, about 30:1, about 20:1, about 15:1, about 10:1, about 5:1, about 2:1, about 1:1, about 1:2, about 1:5, about 1:10, about 1:15, about 1:20, about 1:30, about 1:40, about 1:50, about 1:60, about 1:70, about 1:80, about 1:90, about 1:100, about 1:200, or about 1:500.

In some cases, the non-natural compositions as disclosed herein does not comprise a full-spectrum terpene profile of a cannabis plant. For example, as compared to a control cannabis plant, the non-natural composition may lack at least one type of terpene compound (e.g., at least 1, 2, 3, 4, 5, or more types of terpene compounds normally found in the control cannabis plant may be missing in the non-natural composition). Alternatively, substantially all types of terpene compounds naturally present in the control cannabis plant may be found in the non-natural composition.

In some cases, the non-natural composition comprising the plurality of terpene compound types may comprise an emulsion (e.g., an emulsion of a plurality of droplets or particles as disclosed herein). The plurality of droplets may comprise at least one of the plurality of terpene compound types. At least 1, 2, 3, 4, 5, 6, 7, 8, 9, 10, or more of the plurality of terpene compound types may be encapsulated in the emulsion. At most 10, 9, 8, 7, 6, 5, 4, 3, 2, or 1 of the plurality of terpene compound types may be encapsulated in the emulsion. The plurality of terpene compound types may be encapsulated in the same droplet. Alternatively, the plurality of terpene compound types may be encapsulated in different droplets.

In some cases, the non-natural composition comprising the plurality of terpene compound types may further comprise at least one cannabinoid compound (e.g., at least 1, 2, 3, 4, 5, 6, 7, 8, 9, 10, or more different types of cannabinoid compound). The at least one cannabinoid compound may be encapsulated in a same droplet as at least one of the plurality of terpene compound types. Alternatively or in addition to, the at least one cannabinoid compound may be encapsulated in a droplet that does not comprise any terpene compound type of the plurality of terpene compound types. Alternatively or in addition to, the at least one cannabinoid compound may not be encapsulated.

Compositions (e.g., therapeutic compositions) of the present disclosure may comprise one or more compounds, e.g., one or more encapsulated terpene compounds, cannabinoid compounds, etc. In some embodiments, the one or more compounds as disclosed herein may be encapsulated in a particle (e.g., a population of microparticles or microcapsules having an average diameter of less than or equal to about 1,000 micrometers) comprising the one or more compounds. The one or more compounds may be encapsulated in the particle, such that the one or more compounds are contained within the particle (e.g., within an inner portion of the particle, within one or more shells of the particle) or coupled to the surface (e.g., attached to a surface of the particle). Non-limiting examples of the particle may include an emulsion, a microparticle, a microcapsule, a nanoparticle, a nanocapsule, a colloid, colloid encapsulations, liposomes, etc.

Cannabinoids can be present in a composition (e.g., a food or beverage product, any therapeutic composition) in a quantity of at least about 0.1, 0.2, 0.3, 0.4, 0.5, 0.6, 0.7, 0.8, 0.9, 1, 90, 100, 150, 200, 250, 300, 350, 400, 450, or 500 milligrams (mg). Cannabinoids can be present in a composition in a quantity of at most about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, or 500 milligrams (mg). Cannabinoids can be present in a composition in a quantity of about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, or 500 milligrams (mg). Cannabinoids can be present in a composition in a quantity of from about 50 to about 150 milligrams. Cannabinoids can be present a composition in a quantity of at least about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of the product. Cannabinoids can be present in a composition in a quantity of at most about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of the product. Cannabinoids can be present a composition in a quantity of about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of the product.

Encapsulated cannabinoids can be present in a quantity of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, or 50 micrograms per particle (e.g., microcapsule). Encapsulated cannabinoids can be present in a quantity of at most about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, or 50 micrograms per particle (e.g., microcapsule). Encapsulated cannabinoids can be present in a quantity of about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, or 50 micrograms per particle (e.g., microcapsule). Encapsulated cannabinoids can be present in a quantity of from about 1 to about 10 micrograms per particle (e.g., microcapsule). Encapsulated cannabinoids can be present in a quantity of at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of a particle (e.g., microcapsule). Encapsulated cannabinoids can be present in a quantity of at most about 0.1%, 0.2%, 0.3%, 0.4%, 0.5%, 0.6%, 0.7%, 0.8%, 0.9%, 1%, 2%, 3%, 30%, 35%, 40%, 45%, or 50% by weight of a particle (e.g., microcapsule). Encapsulated cannabinoids can be present in a quantity of about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of a particle (e.g., microcapsule).

The compositions of the present disclosure can comprise one or more terpene compounds, including but not limited to terpenoids such as monoterpenoids, sesquiterpenoids, diterpenoids, and triterpenoids. Terpenes can be acyclic, monocyclic, or polycyclic. Terpenes can include but are not limited to myrcene, limonene, linalool, trans-ocimene, cis-ocimene, alpha-pinene, beta-pinene, alpha-humulene (alpha-caryophyllene), beta-caryophyllene, delta-3-carene, trans-gamma-bisabolene, cis-gamma-bisabolene, trans-alpha-farnesene, cis-beta-farnesene, beta-fenchol, beta-phellandrene, guajol, alpha-gualene, alpha-eudesmol, beta-eudesmol, gamma-eudesmol, terpinolene, alpha-selinene, beta-selinene, alpha-terpincol, fenchone, camphene, cis-sabinene hydrate, alpha-trans-bergamotene, alpha-cis-bergamotene, borneol, gamma-curcumene, alpha-thujene, epi-alpha-bisabolol, ipsdienol, alpha-ylangene, beta-elemene, gamma-muurolene, alpha-cadinene, alpha-longipinene, caryophyllene oxide, beta-citronellene, and combinations thereof.

Terpene compounds can be present in a composition (e.g., a food or beverage product, any therapeutic composition) in a quantity of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, or 500 milligrams (mg). Terpene compounds can be present in a composition in a quantity of at most about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, or 500 milligrams (mg). Terpene compounds can be present in a composition in a quantity of about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, or 500 milligrams (mg). Terpene compounds can be present in a composition in a quantity of from about 50 to about 150 milligrams. Terpene compounds can be present a composition in a quantity of at least about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of the product. Terpene compounds can be present in a composition in a quantity of at most about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of the product. Terpene compounds can be present a composition in a quantity of about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of the product.

Encapsulated terpene compounds can be present in a quantity of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, or 50 micrograms per particle (e.g., microcapsule). Encapsulated terpene compounds can be present in a quantity of at most about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, or 50 micrograms per particle (e.g., microcapsule). Encapsulated terpene compounds can be present in a quantity of about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, or 50 micrograms per particle (e.g., microcapsule). Encapsulated terpene compounds can be present in a quantity of from about 1 to about 10 micrograms per particle (e.g., microcapsule). Encapsulated terpene compounds can be present in a quantity of at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of a particle (e.g., microcapsule). Encapsulated terpene compounds can be present in a quantity of at most about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of a particle (e.g., microcapsule). Encapsulated terpene compounds can be present in a quantity of about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of a particle (e.g., microcapsule).

The compositions of the present disclosure can comprise one or more essential oils or essential oil compounds. Essential oils can include, but are not limited to: Linalool; B-Caryophyllene; B-Myrcene; D-Limonene; Humulene; a-Pinene; Ylang Ylang (Cananga odorata); Yarrow (Achillea millefolium); Violet (Viola odorata); Vetiver (Vetiveria zizanoides); Vanilla (Vanilla plantifolia); Tuberose (Polianthes tuberosa); Thyme (Thymus vulgaris L.); Tea Tree (Melaleuca alternifolia); Tangerine (Citrus reticulata); Spruce, Black (Picea mariana); Spruce (Tsuga Canadensis); Spikenard (Nardostachys jatamansi); Spearmint (Mentha spicata); Sandalwood (Santalum spicatum); Rosewood (Aniba rosacodora); Rosemary Verbenone (Rosmarinus officinalis); Rosemary (Rosmarinus officinalis); Rose (Rosa damascena); Rose Geranium (Pelargonium roseum); Ravensara (Ravensara aromatica); Plai (Zingiber cassumunar) Pine Needle (Pinus sylvestris L.); Petitgrain (Citrus aurantium); Peppermint (Mentha piperita); Pepper, Black (Piper nigrum L.); Patchouli (Pogostemon cablin); Palo Santo (Bursera graveolens); Palmarosa (Cymbopogon martini); Osmanthus (Osmanthus fragrans); Oregano (Origanum vulgare); Orange, Sweet (Citrus sinensis); Oak Moss (Evernia prunastri); Nutmeg (Myristica fragrans) Niaouli (Melaleuca viridifloria); Neroli (aka Orange Blossom) (Citrus aurantium); Myrtle (Myrtus communis); Myrrh (Commiphora myrrha); Mimosa (Acacia decurrens); Melissa (Melissa officinalis L.); Marjoram, Sweet (Origanum majorana); Manuka (Leptospermum scoparium); Mandarin, Red (Citrus deliciosa); Mandarin (Citrus deliciosa); Lotus, White (Nelumbo nucifera); Lotus, Pink (Nelumbo nucifera); Lotus, Blue (Nelumbo nucifera); Lime (Citrus aurantifolia); Lily (Lilum aurantum); Lemongrass (Cymbopogon citratus); Lemon (Citrus limonum); Lavender (Lavandula angustifolium); Lavandin (Lavandula hybrida grosso); Kanuka (Kunzea ericoides); Juniper Berry (Juniperus cummunis); Jasmine (Jasminum officinale); Jasmine Abs (Jasminum sambac); Helichrysum (Helichrysum italicum); Grapefruit, White (Citrus x paradisi); Grapefruit, Pink (Citrus paradisi); Ginger (Zingiber officinalis); Geranium (Pelargonium graveolens); Geranium, Bourbon (Pelargonium graveolens, Herit); Gardenia (Gardenia jasminoides); Galbanum (Ferula galbaniflua); Frankincense (Boswellia carterii); Frangipani (Plumeria alba); Fir Needle White (Abies alba); Fir Needle Siberia (Abies siberica); Fir Needle Canada (Abies balsamca); Fennel, Sweet (Foeniculum vulgare); Eucalyptus Smithii. Eucalyptus Radiata, Eucalyptus Globulus, Eucalyptus Citriodora, Eucalyptus Bluc Mallee (Eucalyptus polybractea); Elemi (Canarium luzonicum); Dill (Anethum graveolens); Cypress (Cupressus sempervirens); Cumin (Cuminum cyminum); Coriander (Coriandum sativum); Cocoa (Theobroma cacao); Clove (Eugenia caryophylatta); Clary Sage (Salvia sclarca); Cistus (aka Labdanum) (Cistus ladaniferus L.); Cinnamon (Cinnamomum zeylanicum); Chamomile, Roman (Anthemis nobilis); Chamomile, Bluc (Matricaria chamomilla); Celery Seed (Apium graveolins); Cedarwood, Western Red (Thuja plicata); Cedarwood, Blood (Juniperus virginiana); Cedarwood Atlas (Cedrus atlantica); Carrot Seed (Daucus carota); Cardamon (Elettaria cardamomum); Caraway Seed (Carum carvi); Cajeput (Melaleuca cajuputi); Cade (Juniperus oxycedrus); Birch, White (Betula alba); Birch, Sweet (Betula lenta); Bergamot (Citrus bergamia); Bay Laurel (Laurus nobilis); Basil (Ocimum basilicum); Basil, Holy (Ocimum sanctum); Basil (Ocimum basilicum); Balsam Poplar (Populus balsamifera); Balsam Peru (Myroxylon balsamum); Angelica (Angelica archangelica L.); and combinations thereof.

Essential oils can be present in a composition (e.g., a food or beverage product, any therapeutic composition) in a quantity of at least about 0.1, 0.2, 0.3, 0.4, 0.5, 0.6, 0.7, 0.8, 0.9, 1, 90, 100, 150, 200, 250, 300, 350, 400, 450, or 500 milligrams (mg). Essential oils can be present in a composition in a quantity of at most about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, or 500 milligrams (mg). Essential oils can be present in a composition in a quantity of about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, or 500 milligrams (mg). Essential oils can be present in a composition in a quantity of from about 50 to about 150 milligrams. Essential oils can be present a composition in a quantity of at least about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of the product. Essential oils can be present in a composition in a quantity of at most about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of the product. Essential oils can be present a composition in a quantity of about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of the product.

Encapsulated essential oils can be present in a quantity of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, or 50 micrograms per particle (e.g., microcapsule). Encapsulated essential oils can be present in a quantity of at most about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, or 50 micrograms per particle (e.g., microcapsule). Encapsulated essential oils can be present in a quantity of about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 45, or 50 micrograms per particle (e.g., microcapsule). Encapsulated essential oils can be present in a quantity of from about 1 to about 10 micrograms per particle (e.g., microcapsule). Encapsulated essential oils can be present in a quantity of at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of a particle (e.g., microcapsule). Encapsulated essential oils can be present in a quantity of at most about 0.1%, 0.2%, 0.3%, 0.4%, 0.5%, 0.6%, 0.7%, 0.8%, 0.9%, 1%, 2%, 3%, 30%, 35%, 40%, 45%, or 50% by weight of a particle (e.g., microcapsule). Encapsulated essential oils can be present in a quantity of about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% by weight of a particle (e.g., microcapsule).

Subjects of the present disclosure can include humans and other animals, such as pets (e.g., dogs, cats, birds, small mammals, snakes) and livestock or farm animals (e.g., cows, pigs, horses, sheep, chickens). Compositions of the present disclosure can be useful for veterinary applications.

The present disclosure provides a unit dose of a composition that may comprise an encapsulated terpene compound. The composition may comprise (e.g., in addition to the encapsulated terpene compound) one or more cannabinoids (e.g., carboxylated or decarboxylated). For example, the composition may comprise a mixture of carboxylated cannabinoids and decarboxylated cannabinoids and one or more terpene compounds. The composition may comprise decarboxylated cannabinoids (e.g. 49 tetrahydrocannabinol) and carboxylated cannabinoids (e.g. tetrahydrocannabinolic acid).

A dose of the present disclosure, which can be a unit dose, can comprise more than about 1 milligram (mg), 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 30 mg, 40 mg, 50 mg, 60 mg, 70 mg, 80 mg, 90 mg, or 100 mg of at least one terpene compound. A dose of a composition of the present disclosure, which can be a unit dose, can comprise about 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 30 mg, 40 mg, 50 mg, 60 mg, 70 mg, 80 mg, 90 mg, or 100 mg of at least one terpene compound. A dose of a composition of the present disclosure, which can be a unit dose, can comprise less than about 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 30 mg, 40 mg, 50 mg, 60 mg, 70 mg, 80 mg, 90 mg, or 100 mg of at least one terpene compound.

A dose of the present disclosure, which can be a unit dose, can comprise more than about 1 milligram (mg), 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 30 mg, 40 mg, 50 mg, 60 mg, 70 mg, 80 mg, 90 mg, or 100 mg of at least one cannabinoid compound. A dose of a composition of the present disclosure, which can be a unit dose, can comprise about 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 30 mg, 40 mg, 50 mg, 60 mg, 70 mg, 80 mg, 90 mg, or 100 mg of at least one cannabinoid compound. A dose of a composition of the present disclosure, which can be a unit dose, can comprise less than about 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 30 mg, 40 mg, 50 mg, 60 mg, 70 mg, 80 mg, 90 mg, or 100 mg of at least one cannabinoid compound.

A unit dose of a composition of the current disclosure can result in a blood concentration, blood plasma concentration, or blood content of a compound in the composition certain amount after a given period of time. A unit dose of a composition may result in a blood content that may be measure from a sample of blood, a sample of blood plasma, a urine sample, a saliva swab, the subject's breath, or other samples of bodily fluids.

A unit dose of an encapsulated terpene compound can result in a blood concentration of the terpene compound of at least about 0.5, 0.75, 1, 1.25, 1.5, 1.75, 2, 2.5, 3, 4, 5, 10, 20, 30, 40, 50, 60, 70, 80, 90, 100, 110, 120, 130, 140, 150, 160, 170, 180, 190, 200, 300, 400, 500 nanograms per milliliter (ng/mL) or greater. Alternatively, the unit dose of the encapsulated terpene compound can result in a blood concentration of the terpene compound of at most about 500, 400, 300, 200, 190, 180, 170, 160, 150, 140, 130, 120, 110, 100, 90, 80, 70, 60, 50, 40, 30, 20, 10, 9, 8, 7, 6, 5, 4, 3, 2, 1, or 0.5 ng/mL. A unit dose of an encapsulated terpene compound can result in a blood concentration of the terpene compound of about at least about 10, 20, 30, 40, 50, 60, 70, 80, 90, 100 ng/ml or higher. A unit dose of an encapsulated terpene compound can result in a blood concentration of the terpene compound from 10 to 200 ng/mL, from 50 to 190 ng/ml, or from 90 to 170 ng/mL.

A daily dose of an encapsulated terpene compound can result in a blood concentration of the terpene compound of at least about 0.5 ng/ml, 0.75 ng/ml, 1 ng/ml, 1.25 ng/ml, 1.5 ng/ml, 1.75 ng/ml, 2 ng/mL, 2.5 ng/ml, 3 ng/mL, 4 ng/ml, 5 ng/mL, 10 ng/ml, 20 ng/ml, 30 ng/mL, 40 ng/mL, 50 ng/mL, 60 ng/mL, 70 ng/mL, 80 ng/mL, 90 ng/mL, 100 ng/ml, 110 ng/mL, 120 ng/mL, 130 ng/mL, 140 ng/mL, 150 ng/ml, 160 ng/mL, 170 ng/mL, 180 ng/ml, 190 ng/mL, 200 ng/ml, 300 ng/mL, 400 ng/mL, 500 ng/mL or higher. A unit dose of an encapsulated terpene compound can result in a blood concentration of the encapsulated terpene compound from 10 to 200 ng/ml, from 50 to 190 ng/ml, or from 90 to 170 ng/mL.

Blood levels of a terpene compound may peak about 1, 2, 3, 4, 5, 6, 7, 8, 9, 10, 12, 14, 16, 18, or 20 minutes after the first dose of an encapsulated terpene compound. Blood levels of a terpene compound may remain detectable for about 1 minute, 2 minutes, 5 minutes, 10 minutes, 1 hour, 2 hours, 3 hours, 4 hours, 6 hours, 8 hours, 10 hours, 12 hours, 18 hours, 24 hours, or 36 hours after the last dose of the encapsulated terpene compound.

A unit dose of a cannabinoid can result in a blood concentration of the cannabinoid of at least about 0.5, 0.75, 1, 1.25, 1.5, 1.75, 2, 2.5, 3, 4, 5, 10, 20, 30, 40, 50, 60, 70, 80, 90, 100, 110, 120, 130, 140, 150, 160, 170, 180, 190, 200, 300, 400, 500 nanograms per milliliter (ng/mL) or greater. Alternatively, the unit dose of the cannabinoid can result in a blood concentration of the cannabinoid of at most about 500, 400, 300, 200, 190, 180, 170, 160, 150, 140, 130, 120, 110, 100, 90, 80, 70, 60, 50, 40, 30, 20, 10, 9, 8, 7, 6, 5, 4, 3, 2, 1, or 0.5 ng/mL. A unit dose of a cannabinoid can result in a blood concentration of the cannabinoid of about at least about 10, 20, 30, 40, 50, 60, 70, 80, 90, 100 ng/ml or higher. A unit dose of a cannabinoid can result in a blood concentration of the cannabinoid from 10 to 200 ng/ml, from 50 to 190 ng/ml, or from 90 to 170 ng/mL.

A daily dose of a cannabinoid can result in a blood concentration of the cannabinoid of at least about 0.5 ng/ml, 0.75 ng/ml, 1 ng/mL, 1.25 ng/mL, 1.5 ng/ml, 1.75 ng/ml, 2 ng/mL, 2.5 ng/ml, 3 ng/mL, 4 ng/mL, 5 ng/mL, 10 ng/mL, 20 ng/ml, 30 ng/ml, 40 ng/ml, 50 ng/mL, 60 ng/mL, 70 ng/mL, 80 ng/mL, 90 ng/mL, 100 ng/mL, 110 ng/ml, 120 ng/mL, 130 ng/mL, 140 ng/mL, 150 ng/mL, 160 ng/mL, 170 ng/mL, 180 ng/ml, 190 ng/mL, 200 ng/ml, 300 ng/ml, 400 ng/mL, 500 ng/mL or higher. A unit dose of a cannabinoid can result in a blood concentration of the cannabinoid from 10 to 200 ng/ml, from 50 to 190 ng/mL, or from 90 to 170 ng/mL.

Blood levels of a cannabinoid may peak about 1, 2, 3, 4, 5, 6, 7, 8, 9, 10, 12, 14, 16, 18, or 20 minutes after the first dose of a cannabinoid. Blood levels of a cannabinoid may remain detectable for about 1 minute, 2 minutes, 5 minutes, 10 minutes, 1 hour, 2 hours, 3 hours, 4 hours, 6 hours, 8 hours, 10 hours, 12 hours, 18 hours, 24 hours, or 36 hours after the last dose of the cannabinoid.

A composition of the current disclosure may be used in a combination therapy. A combination therapy may be administered by a combination treatment regimen. A combination treatment regimen may encompass treatment regimens in which administration of a compound described herein, or a pharmaceutically acceptable salt thereof, is initiated prior to, during, or after treatment with a second agent, and may continue until any time during treatment with the second agent or after termination of treatment with the second agent. The second agent being used in combination may be administered simultaneously or at different times and/or at decreasing or increasing intervals during the treatment period than the first agent.

A combination composition can be formulated to achieve a given, desired or predetermined molar ratio or mass ratio between two or more compounds of the composition. The molar ratio can be adjusted to account for the bioavailability, the uptake, and the metabolic processing of the one or more components of a combination composition. For example, if the bioavailability of a component is low, then the molar amount of a that component can be increased relative to other components in the combination composition. The circulating molar or mass ratio may be achieved within about 0.1, 0.5, 0.75, 1, 3, 5, or 10, 12, 24, or 48 hours after administration. The circulating molar or mass ratio can be maintained for a time period of about or greater than about 0.1, 1, 2, 5, 10, 12, 18, 24, 36, 48, 72, or 96 hours.

### Terpene Profile Determination

In an aspect, the present disclosure provides a method of generating a cannabinoid formulation. In some embodiments, the method comprises determining a terpene profile of a cannabis strain. The terpene profile may comprise at least a first terpene compound and a second terpene compound. The method may further comprise providing a first amount of the first terpene compound, a second amount of the second terpene compound, and a third amount of a cannabinoid compound to a reaction vessel. In the reaction vessel, a weight ratio of the first terpene compound to the second terpene compound may be substantially equivalent to a weight ration of the first terpene compound to the second terpene compound in the terpene profile of the cannabis strain, as previously determined. The method may further comprise, in the reaction vessel, mixing the first terpene compound, the second terpene compound, and the cannabinoid compound to generate an emulsion comprising the first terpene compound, the second terpene compound, and the cannabinoid compound.

In other embodiments, the terpene profile comprises at least 2 terpenes, 3 terpenes, 4 terpenes, 5 terpenes, 6 terpenes, 7 terpenes, 8 terpenes, 9 terpenes, 10 terpenes, 15 terpenes, 20 terpenes, 25 terpenes, 30 terpenes, 35 terpenes, 40 terpenes, 50 terpenes, or more. In some embodiments the method comprises providing 2 terpenes, 3 terpenes, 4 terpenes, 5 terpenes, 6 terpenes, 7 terpenes, 8 terpenes, 9 terpenes, 10 terpenes, 15 terpenes, 20 terpenes, 25 terpenes, 30 terpenes, 35 terpenes, 40 terpenes, 50 terpenes, or more to the reaction vessel. In some embodiments, the resulting emulsion comprises at least 2 terpenes, at least 3 terpenes, at least 4 terpenes, at least 5 terpenes, at least 6 terpenes, at least 7 terpenes, at least 8 terpenes, at least 9 terpenes, at least 10 terpenes, at least 15 terpenes, at least 20 terpenes, at least 25 terpenes, at least 30 terpenes, at least 35 terpenes, at least 40 terpenes, at least 50 terpenes, or more terpenes.

In some embodiments, the terpene profile of a cannabis strain is determined by analyzing a sample of the cannabis strain via mass spectrometry. In some cases, the spectrometry analysis may be performed on a Dani Master HS-GC system. In some cases, the spectrometry analysis may comprise utilizing a full evaporation technique process coupled to a capillary gas chromatography. The analysis process may comprise a mass detector. The mass detector may operate in time-of-flight mode. The spectrometry may characterize the volatile components (e.g., the terpene compounds) of the sample.

### Dosing Forms

The compositions described herein can be compounded into a variety of different dosage forms. It can be in an oral dosage form. The composition may be used orally, such as, for example, in the form of a tablet, a capsule, a pill, a granule, an emulsion, a gel, a plurality of beads encapsulated in a capsule, a powder, a suspension, a liquid, a semi-liquid, a semi-solid, a syrup, a slurry, a chewable form, caplets, soft gelatin capsules, lozenges or solution. Alternatively, a composition can be formulated for inhalation or for intravenous delivery. The compositions can also be formulated as a nasal spray or for injection when in solution form. Alternatively, the composition can be a liquid composition suitable for oral consumption.

A composition may also be formulated onto a solid or semi-solid support. The composition may be formulated on or in a polymeric material (e.g., silicone) and can be used as an injectable polymeric material (e.g., silicone) to prevent blood loss during traumatic injury or surgery. The polymeric material may be a biopolymer. The biopolymer may biodegradable or absorbable into the subject over a period of time.

The polymeric material may facilitate wound repair, assist in a decrease in perceived pain by the subject, exhibit antimicrobial properties, such as slowing the growth of microorganisms, or facilitate overall homeostatic balance in or around the wound or in the subject as a whole. The polymeric material may decrease shock, trauma, or oxidative stress to the area of or around the wound or in subject overall. The polymeric material may also be used as a vehicle for delivering therapeutic material. In some cases, a composition of the current disclosure and a second therapeutic material may be formulated onto a polymeric material that is use before, during, or after a surgical procedure or trauma.

The composition may be formulated for use during and after a surgical procedure, such as onto a medical device. The medical device may be a suture, a plug, thread, an implant, or a prosthetic. The composition may be formulated onto a material that is biodegradable or absorbable and may degrade within the body after at least about 1 day, 2 days, 3 days, 7 days, 1 month, 2 months, or more. Alternatively, the medical device (e.g. suture or plug) may be non-biodegradable or non-absorbable. The composition may facilitate a slow-release of compounds of the composition, which may be desired. The compositions can be formulated onto a medical device that is implanted into a subject during surgery, and may release one or more components over a time period of 1, 4, 6, 8, 12, 16, 20, 24, 36, 48 or more hours.

A composition described herein may be used to enhance the success or results of an implant or prosthetic procedure. For example, a composition may be administered before, during, or after an implant procedure. Implants may be used to replace a missing biological structure, support damaged structure, or enhance existing structure. In some embodiments, an implant may be subdermal. In some embodiments, an implant may be transdermal. Implants may include, for example, cardiovascular implants, orthopedic implants, contraception implants, cosmetic implants, prosthetic limbs, and ocular implants. In some cases, an implant may be a neural lace, and may be implanted into the head cavity, and may be in or near the brain. In some cases, a composition described herein may provide benefits for neuroplasticity and may positively alter the ability of the brain to change over time. Compositions formulated for inhalation can be packaged in an inhaler or nebulizer. An inhaler can be designed to dispense 0.25, 0.5, or 1 unit dose per inhalation. An inhaler can have a canister that holds the subject composition formulated for inhalation, a metering valve that allows for a metered quantity of the formulation to be dispensed with each actuation, and an actuator or mouthpiece that allows for the device to be operated and direct the subject composition into the subject's lungs. The formulated composition can include a liquefied gas propellant and possibly stabilizing excipients. The actuator can have a mating discharge nozzle that connects to the canister and a dust cap to prevent contamination of the actuator. Upon actuation, the subject composition can be volatized, which results in the formation of droplets of the subject composition. The droplets can rapidly evaporate resulting in micrometer-sized particles that may be then inhaled by the subject.

Compositions of the present disclosure suitable for administration (e.g., oral, intravenous, or intramuscular administration) can be in the form of capsules, cachets, tablets, aerosol sprays, powders, granules, gels, syrups, slurries, wafers, lozenge, aqueous compositions, liquid compositions, suspensions in an aqueous or non-aqueous liquid, oil-in-water emulsions, water-in-oil liquid emulsions, colloids, monolayer droplets, micellular droplets, self-emulsifying drug delivery systems (SEDDS), colloid encapsulations (e.g., a single or multi-layer(s) colloid encapsulation), liposomes, colloidosomes, etc. In some cases, oral administration of a colloid encapsulation (e.g., a single layer colloid encapsulation) comprising any of the compositions disclosed herein may enable rapid onset, rapid uptake, and/or enhanced bioavailability, thereby providing an casier administration for subjects in need thereof (e.g., patients) route than other methods.

Applicant has discovered that monolayer droplets, colloidal suspension droplets, or micellular droplets may enhance, modulate, increase, or otherwise enable the effects of a weight ratio of terpenes accordingly to a determined, strain-specific terpene profile. In some cases, the benefit of monolayer droplets, colloidal suspension droplets, or micellular droplets to enhance, modulate, increase, or otherwise enable the effects of a weight ratio of terpenes accordingly to a determined, strain-specific terpene profile may be greater than a bilayer liposome of a similar or equivalent composition.

Compositions of the present disclosure suitable for oral administration can be presented as discrete dosage forms, such as capsules, cachets, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion, including liquid dosage forms (e.g., a suspension or slurry), and oral solid dosage forms (e.g., a tablet or bulk powder). Oral dosage forms can be formulated as tablets, pills, dragees, capsules, emulsions, lipophilic and hydrophilic suspensions, liquids, gels, syrups, slurries, suspensions, for oral ingestion by an individual, patient, or subject to be treated. Such dosage forms can be prepared by any of the methods of formulation. For example, the active ingredients can be brought into association with a carrier, which constitutes one or more necessary ingredients. Capsules suitable for oral administration include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, in some cases, stabilizers. The composition for oral use may be obtained by mixing a composition comprising a solid excipient, in some cases grinding a resulting mixture, and processing the mixture of granules, after adding suitable auxiliaries, if desired, to obtain tablets or dragee cores. Excipients may be fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). Compositions that are prepared may be prepared uniformly and intimately admixing the active ingredient with liquid carriers or finely divided solid carriers or both, and then, if necessary, shaping the product into the desired presentation. For example, a tablet can be prepared by compression or molding, in some cases with one or more accessory ingredients. Compressed tablets can be prepared by compressing in a suitable machine the active ingredient in a free-flowing form, such as powder or granules, in some cases mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

The liquid forms, in which the formulations disclosed herein can be incorporated for administration orally or by injection, include aqueous solution, suitably flavored syrups, aqueous or oil suspensions, and flavored emulsions with edible oils such as cottonseed oil, sesame oil, coconut oil, or peanut oil as well as elixirs and similar pharmaceutical vehicles. Suitable dispersing or suspending agents for aqueous suspensions include synthetic natural gums, such as tragacanth, acacia, alginate, dextran, sodium carboxymethyl cellulose, methylcellulose, polyvinylpyrrolidone or gelatin.

Liquid preparations for oral administration can take the form of, for example, solutions, syrups or suspensions, or they can be presented as a dry product for reconstitution with water or other suitable vehicles before use. Such liquid preparations can be prepared by conventional approaches with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); preservatives (e.g., methyl or propyl p-hydroxybenzoates or sorbic acid); and artificial or natural colors and/or sweeteners.

This disclosure further encompasses anhydrous compositions and dosage forms comprising an active ingredient, since water can facilitate the degradation of some compounds. For example, water can be added (e.g., 5%) to simulate long-term storage in order to determine characteristics such as shelf-life or the stability of formulations over time. Anhydrous compositions and dosage forms of the present disclosure can be prepared using anhydrous or low moisture containing ingredients and low moisture or low humidity conditions. Compositions and dosage forms of the present disclosure which contain lactose can be made anhydrous if substantial contact with moisture and/or humidity during manufacturing, packaging, and/or storage is expected. An anhydrous composition can be prepared and stored such that its anhydrous nature is maintained. Accordingly, anhydrous compositions can be packaged using materials that prevent exposure to water such that they can be included in suitable formulary kits. Examples of suitable packaging include, but are not limited to, hermetically sealed foils, plastic, unit dose containers, blister packs, and strip packs.

An ingredient described herein can be combined in an intimate admixture with a pharmaceutical carrier according to conventional pharmaceutical compounding techniques. The carrier can take a wide variety of forms depending on the form of preparation desired for administration. In preparing the compositions for an oral dosage form, pharmaceutical media can be employed as carriers, such as, for example, water, glycols, oils, alcohols, flavoring agents, preservatives, coloring agents, in the case of oral liquid preparations (such as suspensions, solutions, and elixirs) or acrosols; or carriers such as starches, sugars, micro-crystalline cellulose, diluents, granulating agents, lubricants, binders, and disintegrating agents can be used in the case of oral solid preparations, with or without employing the use of lactose. For example, suitable carriers include powders, capsules, and tablets, with the solid oral preparations. If desired, tablets can be coated by standard aqueous or nonaqueous techniques.

Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, cthyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Binders suitable for use in dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Lubricants which can be used to form compositions and dosage forms of the present disclosure include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethylaureate, agar, or mixtures thereof. Additional lubricants include, for example, a syloid silica gel, a coagulated acrosol of synthetic silica, or mixtures thereof. A lubricant may be added, such as, for example, in an amount of less than about 1 weight percent of the composition.

Lubricants can be also be used in conjunction with tissue barriers which include, but are not limited to, polysaccharides, polyglycans, seprafilm, interceed and hyaluronic acid.

Disintegrants can be used in the compositions of the present disclosure to provide tablets that disintegrate when exposed to an aqueous environment. Too much of a disintegrant can produce tablets which can disintegrate in the bottle. Too little can be insufficient for disintegration to occur and can thus alter the rate and extent of release of the active ingredient(s) from the dosage form. Thus, a sufficient amount of disintegrant that is neither too little nor too much to detrimentally alter the release of the active ingredient(s) can be used to form the dosage forms of the compounds disclosed herein. The amount of disintegrant used can vary based upon the type of formulation and mode of administration, and can be readily discernible to those of ordinary skill in the art. About 0.5 to about 15 weight percent of disintegrant, or about 1 to about 5 weight percent of disintegrant, can be used in the pharmaceutical composition. Disintegrants that can be used to form compositions and dosage forms of the present disclosure include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Examples of suitable fillers for use in the compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

In some cases, aqueous suspensions and/or elixirs may be desired for oral administration. In such cases, the active ingredient therein may be combined with various sweetening or flavoring agents, coloring matter or dyes and, if desired, emulsifying and/or suspending agents, together with diluents such as water, ethanol, propylene glycol, glycerin or various combinations thereof.

The tablets can be uncoated or coated to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

In one embodiment, the composition can include a solubilizer to ensure good solubilization and/or dissolution of the compound of the present disclosure and to minimize precipitation of the compound of the present disclosure. This can be especially important for compositions for non-oral use, e.g., compositions for injection. A solubilizer can also be added to increase the solubility of the hydrophilic drug and/or other components, such as surfactants, or to maintain the composition as a stable or homogeneous solution or dispersion.

The composition can further include one or more pharmaceutically acceptable additives or pharmaceutically acceptable excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof. A non-exhaustive list of examples of excipients includes monoglycerides, magnesium stearate, modified food starch, gelatin, microcrystalline cellulose, glycerin, stearic acid, silica, yellow beeswax, lecithin, hydroxypropylcellulose, croscarmellose sodium, and crospovidone.

In some embodiments, a compound or composition described herein may be formulated or administered in combination with another active ingredient or ingredients. In some cases, a cannabinoid composition may be administered with psychedelic compounds for therapeutic enhancement. The therapeutic enhancement may be via optimization of the endocannabinoid system, neuroplasticity, neural trimming, anti-psychotic effects, anxiety effects, enhanced neurogenesis, or a combination thereof.

In some cases, a cannabinoid composition as described herein may be used in combination with psychedelic compounds, such as 3,4-methylenedioxymethamphetamine (MDMA), psilocybin, lysergic acid diethylamide (LSD). In some cases, a cannabinoid composition may be used in combination with psychedelic assisted therapeutic programs, and may assist in overall efficacy.

The compositions described herein can also be formulated as extended-release, slow-release, sustained-release or time-release such that one or more components are released over time. Compositions of the present disclosure may have half-lives of at least about 1 minute, 10 minutes, 30 minutes, 1 hours, 2 hours, 3 hours, 4 hours, 5 hours, 6 hours, 12 hours, 1 day, 2 days, 3 days, 4 days, 5 days, 6 days, 1 week, 2 weeks, 3 weeks, 4 weeks, or more. Delayed release can be achieved by formulating the one or more components in a matrix of a variety of materials or by microencapsulation (e.g., microencapsulation in a material that has a predetermined rate of degradation, or a material with pores with pore sizes that permit controllable release). The compositions can be formulated to release one or more components over a time period of 1, 4, 6, 8, 12, 16, 20, 24, 36, or 48 hours. The release of the one or more components can be at a constant or changing rate.

Examples of the at least one therapeutic compound may include, but are not limited to, a nucleic acid, a polynucleotide, an amino acid, a polypeptide, a lipid, a carbohydrate, a small molecule (e.g., at least one cannabinoid compound as disclosed herein), an enzyme, a ribosome, a proteasome, an antibody, a variant thereof, or any combination thereof.

A therapeutic composition may comprise a plurality of therapeutic doses. The plurality of therapeutic doses may be administered to a subject in need thereof via the same route (e.g., via oral administration). Alternatively or in addition to, the plurality of therapeutic doses may be administered to the subject in need thereof via different routes (e.g., a first therapeutic dose via oral administration and a second therapeutic dose via nebulization, intravenous administration, or intramuscular administration). The plurality of therapeutic doses may be administered to the subject at the same time. Alternatively or in addition to, the plurality of therapeutic doses may be administered to the subject at different times.

Droplets (i.e., microcapsules) can be characterized by a size (e.g., a diameter). The droplet size can be about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, 4.5, 5, 5.5, 6, 6.5, 7, 7.5, 8, 8.5, 9, 9.5, 10, 15, 20, 25, 30, 35, 40, 45, 50, 55, 60, 65, 70, 75, 80, 85, 90, 95, 100, 150, 200, 250, 300, 350, 400, 450, or 500 micrometers. The droplet size can be less than or equal to about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, 4.5, 5, 5.5, 6, 6.5, 7, 7.5, 8, 8.5, 9, 9.5, 10, 15, 20, 25, 30, 35, 40, 45, 50, 55, 60, 65, 70, 75, 80, 85, 90, 95, 100, 150, 200, 250, 300, 350, 400, 450, or 500 micrometers. The droplet size can be greater than or equal to about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, 4.5, 5, 5.5, 6, 6.5, 7, 7.5, 8, 8.5, 9, 9.5, 10, 15, 20, 25, 30, 35, 40, 45, 50, 55, 60, 65, 70, 75, 80, 85, 90, 95, 100, 150, 200, 250, 300, 350, 400, 450, or 500 micrometers. The droplet size can be from about 0.1 to about 0.2 micrometers. The droplet size can be from about 0.05 to about 0.25 micrometers. The droplet size can be from about 0.05 to about 0.55 micrometers. The droplet size can be from about 0.05 to about 1 micrometers. The size distribution in a population of droplets can be homogeneous or substantially homogeneous. For example, a population of droplets can be characterized by dispersity, or polydispersity index (PDI), of less than or equal to about 20, 19, 18, 17, 16, 16, 15, 14, 13, 12, 11, 10, 9, 8, 7, 6, 5, 4.9, 4.8, 4.7, 4.6, 4.5, 4.4, 4.3, 4.2, 4.1, 4.0, 3.9, 3.8, 3.7, 3.6, 3.5, 3.4, 3.3, 3.2, 3.1, 3.0, 2.9, 2.8, 2.7, 2.6, 2.5, 2.4, 2.3, 2.2, 2.1, 2.0, 1.9, 1.8, 1.7, 1.6, 1.5, 1.45, 1.40, 1.35, 1.30, 1.25, 1.20, 1.15, 1.14, 1.13, 1.12, 1.11, 1.10, 1.09, 1.08, 1.07, 1.06, 1.05, 1.04, 1.03, 1.02, 1.01, or 1.00.

Any composition disclosed herein may be used to treat or decrease symptoms of a number of classes of disorders as provided in the present disclosure, such as, but not limited to, pain, anti-inflammatory disorders, psychiatric disorders, and sleep disorders.

The disorders as disclosed herein may comprise one or more psychological disorders comprising depression, psychotic disorder, schizophrenia, schizophreniform disorder (acute schizophrenic episode); schizoaffective disorder; bipolar I disorder (mania, manic disorder, manic-depressive psychosis); bipolar II disorder; major depressive disorder with psychotic feature (psychotic depression); delusional disorders (paranoia); Shared Psychotic Disorder (Shared paranoia disorder); Brief Psychotic disorder (Other and Unspecified Reactive Psychosis); Psychotic disorder not otherwise specified (Unspecified Psychosis); paranoid personality disorder; schizoid personality disorder; schizotypal personality disorder, anxiety disorder, panic disorder, panic attacks, agoraphobia, attention deficit syndrome, premenstrual dysphoric disorder (PMDD), and/or premenstrual syndrome (PMS).

A composition described herein can be formulated in as matrix pellets in which particles of the subject composition are embedded in a matrix of water-insoluble plastic and which are enclosed by a membrane of water-insoluble plastic containing embedded particles of lactose, produces and maintains plasma levels of the subject composition within the targeted therapeutic range. A composition can be formulated as a sustained or controlled release capsule or tablet. A sustained or controlled release tablet may be formed by coating core granules composed mainly of the subject composition with a layer of a coating film composed of a hydrophobic material and a plastic excipient and, in some cases, containing an enteric polymer material, to form coated granules and then by compressing the coated granules together with a disintegrating excipient. Such sustained or controlled release capsule or tablet may release the composition in a substantially sustained or controlled manner over a given period of time, such as a substantially constant rate of release over a period of at least 0.1 hour, 0.5 hours, 1 hour, 2 hours, 3 hours, 4 hours, 5 hours, 6 hours, 12 hours, 24 hours, or more. Such sustained or controlled release capsule or tablet may permit at least about 10%, 20%, 30%, 40%, 50%, 60%, 70%, 80%, 90%, 95%, or greater of the composition to be released over a period of at least 0.1 hour, 0.5 hours, 1 hour, 2 hours, 3 hours, 4 hours, 5 hours, 6 hours, 12 hours, 24 hours, or more.

The controlled release formulations may exhibit plasma concentration curves having initial (e.g., from 2 hours after administration to 4 hours after administration) slopes less than 75%, 50%, 40%, 30%, 20% or 10% of those for an immediate release formulation of the same dosage of the same cofactor.

The rate of release of the cofactor as measured in dissolution studies may be less than about 80%, 70%, 60%, 50%, 40%, 30%, 20%, or 10% of the rate for an immediate release formulation of the same cofactor over the first 1, 2, 4, 6, 8, 10, or 12 hours.

The controlled release formulations provided herein can adopt a variety of formats. The formulation may be in an oral dosage form, including liquid dosage forms (e.g., a suspension or slurry), and oral solid dosage forms (e.g., a tablet or bulk powder), such as, but not limited to those, those described herein.

Tablets or pills can also be coated or otherwise compounded to provide a dosage form affording the advantage of prolonged action. For example, the tablet or pill can comprise an inner dosage an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings such materials including a number of polymeric acids and mixtures of polymeric acids with such materials as shellac, cetyl alcohol and cellulose acetate. A formulation comprising a plurality of cofactors can have different cofactors released at different rates or at different times. For example, there can be additional layers of cofactors interspersed with enteric layers.

The compositions can be formulated in a food composition. For example, the compositions can be a beverage or other liquids, solid food, semi-solid food, with or without a food carrier. For example, the compositions can include a black tea supplemented with any of the compositions described herein. The composition can be a dairy product supplemented any of the compositions described herein. The compositions can be formulated in a food composition. For example, the compositions can comprise a beverage, solid food, semi-solid food, or a food carrier.

Liquid food carriers, such as in the form of beverages, such as supplemented juices, coffees, teas, sodas, and flavored waters can be used. For example, the beverage can comprise the formulation as well as a liquid component, such as various deodorant or natural carbohydrates present in conventional beverages. Examples of natural carbohydrates include, but are not limited to, monosaccharides such as, glucose and fructose; disaccharides such as maltose and sucrose; conventional sugars, such as dextrin and cyclodextrin; and sugar alcohols, such as xylitol and crythritol. Natural deodorant such as taumatin, stevia extract, levaudioside A, glycyrrhizin, and synthetic deodorant such as saccharin and aspartame can also be used. Agents such as flavoring agents, coloring agents, and others can also be used. For example, pectic acid and the salt thereof, alginic acid and the salt thereof, organic acid, protective colloidal adhesive, pH controlling agent, stabilizer, a preservative, glycerin, alcohol, or carbonizing agents can also be used. Fruit and vegetables can also be used in preparing foods or beverages comprising the formulations discussed herein.

Alternatively, the compositions can be a snack bar supplemented with any of the compositions described herein. For example, the snack bar can be a chocolate bar, a granola bar, or a trail mix bar. In yet another embodiment, the present dietary supplement or food compositions are formulated to have suitable and desirable taste, texture, and viscosity for consumption. Any suitable food carrier can be used in the present food compositions. Food carriers of the present disclosure include practically any food product. Examples of such food carriers include, but are not limited to food bars (granola bars, protein bars, candy bars, etc.), cereal products (oatmeal, breakfast cereals, granola, etc.), bakery products (bread, donuts, crackers, bagels, pastries, cakes, etc.), beverages (milk-based beverage, sports drinks, fruit juices, alcoholic beverages, bottled waters), pastas, grains (rice, corn, oats, rye, wheat, flour, etc.), egg products, snacks (candy, chips, gum, chocolate, etc.), meats, fruits, and vegetables. In an embodiment, food carriers employed herein can mask the undesirable taste (e.g., bitterness). Where desired, the food composition presented herein exhibit more desirable textures and aromas than that of any of the components described herein. For example, liquid food carriers can be used according to the present disclosure to obtain the present food compositions in the form of beverages, such as supplemented juices, coffees, teas. Solid food carriers can be used according to the present disclosure to obtain the present food compositions in the form of meal replacements, such as supplemented snack bars, pasta, breads. Alternatively, semi-solid food carriers can be used according to the present disclosure to obtain the present food compositions in the form of gums, or chewy candies or snacks.

### Indications

A composition as disclosed herein may be administered (e.g., via one or more unit doses) to a subject (e.g., a subject having or is suspected of having a condition of disease as disease of interest), and the administering may effect one or more members comprising (i) reduced degree (e.g., as measured by a scoring system or by one or more biomarkers from the subject, such as a target molecule, protein, etc.) of a disease or condition of the subject as compared to a control, or (ii) reduction in one or more symptoms of the disease or condition as compared to a control. In some cases, a composition disclosed herein may be administered to a subject, and the control may be (1) one or more states or conditions of the subject prior to the administering, (2) a different subject having or is suspected of having the same disease or condition, who has not been administered with the composition disclosed herein, (3) a different subject having or is suspected of having the same disease or condition, who has been administered with a composing comprising a plurality of compounds at a different ratio (e.g., a different weight ratio), or (4) a healthy subject.

Administering the composition as disclosed herein may reduce the degree of the disease or condition of the subject by at least about 1%, 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or more, as compared to the control.

Administering the composition as disclosed herein may reduce a score for ascertaining the degree of the disease or condition of the subject by at least about 1, 2, 3, 4, 5, 6, 7, 8, 9, 10, 11, 12, 13, 14, 15, 16, 17, 18, 19, 20, 21, 22 23, 24, 25, 26, 27, 28, 29, 30, 35, 40, 45, 50, 55, 60, 65, or more, as compared to the control.

Administering the composition as disclosed herein may reduce a score for ascertaining the degree of the disease or condition of the subject by at least about 1%, 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or more, as compared to the control.

Administering the composition as disclosed herein may reduce a degree of a positive biomarker (e.g., a biomarker whose level is increased due to the condition or disease) by at least about 1%, 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or more, as compared to the control.

Administering the composition as disclosed herein may increase a degree of a negative biomarker (e.g., a biomarker whose level is decreased due to the condition or disease) by at least about 1%, 5%, 10%, 15%, 20%, 25%, 30%, 35%, 40%, 45%, 50%, 55%, 60%, 65%, 70%, 75%, 80%, 85%, 90%, 95%, 100%, 150%, 200%, 250%, 300%, 350%, 400%, 450%, 500%, 1000%, or more, as compared to the control.

Administering the composition as disclosed herein may reduce a degree one or more symptoms of the disease or condition by at least about 1%, 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or more, as compared to the control.

In some examples, the disease or condition may be post-traumatic stress disorder (PTSD). An indication or degree of PTSD may be measured by a PTSD checklist (PCL; a score ranging between 17 and 85) (e.g., PCL-M for military subjects, PCL-C for civilian subjects, PCL-S for subjects with specific stressful experience(s)). One or more symptoms of PTSD may include, but are not limited to, intrusive memories (e.g., recurrent, unwanted distressing memories), avoidance (e.g., avoiding places, activities, or people), mood (e.g., negative mood), etc. Non-limiting examples of a biomarker for PTSD may include Allopregnanolone (e.g., decreased with PTSD), Dehydroepiandrosterone (e.g., increased with PTSD), Dehydroepiandrosterone (e.g., increased with PTSD), NPY hormones (e.g., decreased with PTSD), Endocannabinoids (e.g., decreased in PTSD), etc.

In some examples, the disease or condition may be insomnia. An indication or degree of insomnia may be measured by an insomnia severity index (ISI; a score ranging between 0 and 28). Examples of symptoms of insomnia may include, but are not limited to, difficulty falling asleep, waking up during the night, waking up too carly, fatigue, irritability, depression, anxiety, attention deficit, etc. Non-limiting examples of a biomarker may include Brain-derived neurotrophic factor (BDNF, such as serum BDNF) (e.g., decreased with insomnia).

In some examples, the disease or condition may be an addiction (e.g., alcohol, smoking, drug, etc.). An indication or degree of the addiction may be measured by an addiction severity index (ASI; a score ranging between 0 and 9).

In some examples, the disease or condition may be alcohol addiction. An indication or degree of the alcohol addiction may be measured by Alcohol Use Disorders Identification Test (AUDIT; a score ranging between 0 and 40). Examples of symptoms of alcohol addiction may include, but are not limited to, excessive drinking (e.g., consumption of alcohol above a threshold value, such as greater than or equal to 8 units per week, 9 units per week, 10 units per weck, 11 units per week, 12 units per week, 13 units per weck, 14 units per week, 15 units per weck, 16 units per weck, 17 units per weck, 18 units per week, 19 units per week, 20 units per weck, or more), mood (e.g., negative mood), etc. Non-limiting examples of biomarkers increased due to alcohol addiction may include Gamma-glutamyltransferase (GGT), Alanine aminotransferase (ALT), Aspartate aminotransferase (AST), Carbohydrate-deficient transferrin (CDT), N-acetyl-β-hexosaminidase, Whole blood-associated acetaldehyde (WBAA), Mean corpuscular volume (MCV), Apolipoprotein J, 5-hydroxytryptophol (5-HTOL), Salsolinol, Fatty acid ethyl esters (FAEE), Ethyl glucuronide (EtG), etc.

In some examples, the disease or condition may be a nicotine addiction (e.g., smoking addiction). An indication or degree of the nicotine addiction may be measured by Fagerstrom Test For Nicotine Dependence (FTND; a score ranging between 0 and 10). Examples of symptoms of nicotine addiction may include, but are not limited to, excessive smoking, withdrawal signs (e.g., anxiety, irritability, restlessness, negative mood, frustration, anger, insomnia, etc.), progression of a disease (e.g., cancer, tumor), etc. Non-limiting examples of biomarkers increased due to nicotine addiction may include blood nicotine level.

Terpenoids may have a beneficial effect on a subject. Terpenoids may be formulated into a composition described herein and given to a subject as a nutritional supplement or a dietary supplement. A composition may provide nutrients or compounds that may otherwise not be consumed in sufficient quantities in a normal dict. A composition may contain compounds that may be beneficial to the health, physical wellbeing, and emotional wellbeing of a subject. For example, a composition may be used as a boost of energy.

A composition of the current disclosure may be used to treat or decrease the symptoms of nausea or vomiting. A subject who is administered a unit dose of a composition may observe a decrease in symptoms related to nausea or vomiting.

A composition of the current disclosure may be used to treat an eating disorder, such as anorexia, cachexia, bulimia nervosa, rumination disorder, avoidant or restrictive food intake disorder.

A composition of the current disclosure may be used as a sleep aid or to help with symptoms of insomnia. A composition may help a subject relax, fall asleep faster, improve sleep quantity, or improve sleep quality.

A composition of the current disclosure may be used to mediate, limit, and reverse the effects of oxidative damage or oxidative stress. The imbalance of reactive oxygen species within a subject may be corrected or mediated with administration of a composition described herein.

Oxidative stress may occur prior to, during, and/or after surgery. The oxidative stress may be due to anesthesia used during the surgery, the surgical trauma, the psychological stress associated with surgery, or a combination thereof. A composition may be administered to a subject prior to, during, or after surgery. A composition may be administered to relieve traumatic shock that may be caused by surgery or injury.

Oxidative stress may be caused by a physical stress factor or an emotional stress factor. A composition of the current disclosure may be used to treat post-traumatic stress disorder (PTSD). Some symptoms of PTSD that may be relieved by a composition include: flashbacks, bad dreams, frightening thoughts, avoidance of certain thoughts or feelings, being casily startled, feeling tense, having difficulty sleeping, cognition or mood symptoms such as trouble remember features of the traumatic event, distorted feelings such as guilt or blame, and loss of interest in enjoyable activities.

A composition may be administered to prevent or limit the severity of developing post-traumatic stress disorder. The composition may be administered after physical or emotional stress, such as, for example, after a physical sport, a contact sport, a physical combat, a physical confrontation, a military drill or exercise, and military combat.

A composition of the current disclosure may be used to treat, alleviate, or cease the symptoms of addiction, addicted behavior, physical dependence, or psychological dependence. Addiction may be characterized by compulsive engagement in stimuli. Addiction may be rated based on a severity index, such as the addiction severity index (ASI). The ASI severity ratings may be based on a 10 point scale, from 0-9. A rating of 0-1 may be classified as no real problem, treatment not indicated. A rating of 2-3 may be classified as a light problem, treatment may not be indicated. A rating of 4-5 may be classified as a moderate problem, and some treatment may be indicated. A rating of 6-7 may be considerable a problem, and treatment may be necessary. A rating of 8-9 may be considered an extreme problem, and treatment may be absolutely necessary.

Examples of drug and behavioral addictions include, but are not limited to, alcoholism, cocaine addiction, smoking addiction, nicotine addiction, opiate addiction, food addiction, amphetamine addiction, and gambling addiction. For example, a composition described herein may be used to alleviate alcohol use disorder (AUD).

A composition described herein may be used to alleviate smoking addiction. A composition may be used as part of a smoking cessation program, where a subject is administered tobacco infused with cannabinoids. A composition may also be administered via water soluble methods, to allow for membrane absorption for natural and gradual decrease in addiction. Additionally, a composition described herein may contribute to anti-anxiety effects. For example, a composition administered in water soluble form may provide rapid anti-anxiety effects to curb addition via mucosal membrane absorption. A cannabinoid composition may be part of program to decrease tobacco usage over time.

In some cases, a cannabinoid compound may have multiple bell curves of efficacy. The bell curves of efficacy may change or modulate on a daily basis depending on a number of factors of the subject, including oxidative stress.

A composition of the current disclosure may be administered to a subject during and/or after treatment. A subject may observe a decrease symptoms or a decrease in severity rating according to a severity index scale (e.g. the ASI severity index).

A composition may be used to treat cancer or a tumor. Cancers that are liquid tumors can be those that occur, for example, in blood, bone marrow, and lymph nodes, and can include, for example, leukemia, myeloid leukemia, lymphocytic leukemia, lymphoma, Hodgkin's lymphoma, melanoma, and multiple myeloma. Leukemias include, for example, acute lymphoblastic leukemia (ALL), acute myeloid leukemia (AML), chronic lymphocytic leukemia (CLL), chronic myelogenous leukemia (CML), and hairy cell leukemia. Cancers that are solid tumors include, for example, prostate cancer, testicular cancer, breast cancer, brain cancer, pancreatic cancer, colon cancer, thyroid cancer, stomach cancer, lung cancer, ovarian cancer, Kaposi's sarcoma, skin cancer, squamous cell skin cancer, renal cancer, head and neck cancers, throat cancer, squamous carcinomas that form on the moist mucosal linings of the nose, mouth, throat, bladder cancer, osteosarcoma, cervical cancer, endometrial cancer, esophageal cancer, liver cancer, and kidney cancer. A composition described herein may be used to treat cervical cancer.

A composition described herein may be used to treat prostate cancer. A subject may be evaluated based on a level of prostate-specific antigen, or PSA, a protein produced by prostate gland cells. Elevated blood levels of PSA may be associated with subjects with prostate cancer.

A composition may be administered to a subject that has been diagnosed with prostate cancer. In some cases, a composition may be administered to a subject with a PSA greater than about 1 nanograms per milliliter (ng/mL), 2 ng/ml, 3 ng/ml, 4 ng/mL, 5 ng/ml, or 6 ng/mL, or higher. Administration with a composition may have a dosing holiday after a subject's level of PSA is below a certain threshold. In some cases, a dosing holiday may begin after PSA drops below about 20 nanograms per milliliter (ng/mL), 10 ng/ml, 5 ng/ml, 4 ng/ml, 3 ng/mL, 2 ng/mL, or 1 ng/mL. A dosing holiday of a composition may be substituted with another compound or composition. For example, an amount of delta-9-tetrahydrocannabinol (THC) may be administered during a dosing holiday of a composition described herein. The amount of THC administered may be at most about 50 mg/kg, 40 mg/kg, 30 mg/kg, 20 mg/kg, 10 mg/kg, 5 mg/kg, 4 mg/kg, 3 mg/kg, 2 mg/kg, 1 mg/kg, or less.

A composition of the current disclosure may be used to treat an eating disorder or a weight disorder, such as, for example, anorexia and cachexia. Subjects may observe an increase in appetite after at least a unit dose of a composition of the present disclosure. In some cases, 49 tetrahydrocannabinol in a composition may result in an appetite enhancing effect with a unit dose of at least 1 mg, 2 mg, 3 mg, 4 mg, 5 mg, 10 mg, 15 mg, or 20 mg. An increase in appetite may continue for an extended period of time, such as at least 1 day, 2 days, 5 days, 7 days, 1 week, 2 weeks, 1 month, 3 months, 6 months, or 12 months after the last unit dose of a composition is taken.

A composition of the current disclosure may be used to treat a muscle related disorder or a movement disorder, such as, for example, spasticity, tremor, ataxia, bladder control, Tourette's syndrome, dystonia, Parkinson's disease, Huntington disease, and tardive dyskinesia. Spasticity may have been caused by pain, bone or join deformities, or accidents or injury to the spinal cord.

A composition of the current disclosure may be used to treat pain. The pain may be an acute pain. The pain may be chronic pain. The pain may be associated with a disease. Pain in a subject may be neuropathic pain, menstrual pain, chronic headaches, or back pain. Pain may be due to a disease or a disorder, or may be caused by injury. Pain may be caused by a disease such as cancer, chronic bowel inflammation, neuralgias, damaged nerves, diabetes, multiple sclerosis, an infection, or old age.

Pain can be nociceptive pain (i.e., pain caused by tissue damage), neuropathic pain or psychogenic pain. The pain may be caused by or associated with a disease (e.g., cancer, arthritis, diabetes). Alternatively, the pain is caused by injury (e.g., sports injury, trauma). Non-limiting examples of pain that may be amenable to treatment with the compositions and methods herein include: neuropathic pain including peripheral neuropathy, diabetic neuropathy, post herpetic neuralgia, trigeminal neuralgia, back pain, neuropathy associated with cancer, neuropathy associated with HIV/AIDS, phantom limb pain, carpal tunnel syndrome, central post-stroke pain, pain associated with chronic alcoholism, hypothyroidism, uremia, pain associated with multiple sclerosis, pain associated with spinal cord injury, pain associated with Parkinson's disease, epilepsy, osteoarthritic pain, rheumatoid arthritic pain, visceral pain, and pain associated with vitamin deficiency; and nociceptive pain including pain associated with central nervous system trauma, strains/sprains, and burns; myocardial infarction, acute pancreatitis, post-operative pain, posttraumatic pain, renal colic, pain associated with cancer, pain associated with fibromyalgia, pain associated with carpal tunnel syndrome, and back pain.

A composition of the current disclosure may be used to treat or reduce an effect of a nervous system disease or disorder. Examples of a nervous system disease/disorder may include, but are not limited to, spinal cord damage, stroke, cerebral infarction, and cerebral ischemia. Additional examples of a nervous system disease/disorder may include multiple sclerosis neuropsychiatric diseases, neurological diseases, psychosis, dementia, Alzheimer's disease, Parkinson's disease, Huntington's chorea, dyskinesia, hyperactivity, mania, attention deficit disorder, anxiety, dyslexia, schizophrenia, Tourette syndrome, behavioral disorders, cognitive disorders, etc.

The compositions and methods described herein may be utilized to ameliorate a level of pain in a subject. A level of pain in a subject may be ameliorated by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99% or 100%. A level of pain in a subject can be assessed by a variety of methods. A level of pain may be assessed by self-reporting (i.e., a human subject expresses a verbal report of the level of pain he/she is experiencing). A level of pain may be assessed by behavioral indicators of pain, for example, facial expressions, limb movements, vocalization, restlessness and guarding. These types of assessments may be useful for example when a subject is unable to self-report (e.g., an infant, an unconscious subject, a non-human subject). A level of pain may be assessed after treatment with a composition of the present disclosure as compared to the level of pain the subject was experiencing prior to treatment with the composition.

A composition of the current disclosure may be used to reduce intraocular pressure or fluid pressure in the eye, and may be used to treat a number of diseases associated with abnormal intraocular pressure, including, but not limited to, glaucoma, iritis, retinal detachment. A composition may decrease intraocular pressure by 5%, 10%, 20%, 30%, 40%, 50%, or more.

The methods and compositions of the present disclosure may be utilized to treat epilepsy. Compositions described herein may be used to prevent or control epileptic seizures. Epileptic seizures may be classified as tonic-clonic, tonic, clonic, myoclonic, absence or atonic seizures. The compositions and methods herein may prevent or reduce the number of epileptic seizures experienced by a subject by at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99% or higher.

The methods and compositions of the present disclosure may be utilized to relieve the symptoms of an inflammatory disease of the airways of the lungs, or asthma. The compositions may reduce the severity of asthma symptoms, or change the severity classification, such as from severe persistent, to moderate persistent, to mild persistent, to intermittent.

A composition of the current disclosure may be used to relieve symptoms associated with withdrawal in dependency on alcohol and drugs, such as benzodiazepines and opiates. The composition may relieve withdrawal symptoms such as sleep disturbance, irritability, increased tension, anxiety, panic attacks, tremors, sweating, difficulty concentrating, confusion, memory loss, weight loss or weight gain, headaches, or muscular pains.

A composition of the current disclosure may be used to treat psychiatric disorders, including, but not limited to, sleep disorder, anxiety disorders, panic disorders, obsessive-compulsive disorder, bipolar disorder, depression, mood disorders, personality disorders, psychotic disorders, such as schizophrenia or delusional disorder. A composition may be used to treat a bipolar episode, wherein a symptom may include an unusual shift in mood, energy, activity level, and the inability to carry out day-to-day tasks.

A composition of the current disclosure may be used to treat autoimmune diseases or inflammation, such as, but not limited to, arthritis, lupus, vitiligo, anemia, psoriasis, scleroderma, inflammatory bowel diseases, and type 1 diabetes.

A composition may be used to treat pruritus, ADS (attention deficit syndrome), high blood pressure, tinnitus, chronic fatigue syndrome, and restless leg syndrome.

A composition may be used to treat or relieve the symptoms of the hiccups or synchronous diaphragmatic flutter (SDF). Hiccups may be classified as acute, chronic, persistent, or intractable. In some cases, a compound or composition may be used to treat or alleviate the symptoms of chronic hiccups.

A composition may be used to treat or alleviate the symptoms of menopause or pre-menopause. A composition may decrease the frequency and/or intensity of symptoms that include, for example, hot flashes, night sweats, pain during intercourse, increased anxiety or irritability, and the need to urinate more often.

A composition may be used to treat or sterilize wounds. A composition may be used in conjunction with citric acid, or may be formulated into one composition for use in sterilizing open wounds.

A composition described herein may be used with poison or venom treatment. The composition may be administered before, during, or after administration of a poison antidote or an antivenom. The composition may be administered after exposure to a toxin or poison and may be in the absence of an antidote. Administration for use with a poison antidote may be via injection, sublingual, oral, via nasal spray, or a transdermal patch. Without wishing to be bound by theory, the composition disclosed herein may help protect the tissue, nervous system, and/or assist with regulating overall homeostasis in the subject. The composition disclosed hercin may help decrease oxidative stress, tissue damage, organ damage, or neural trauma. The composition may also enhance cellular protection, health, and overall homeostatic balance.

In some cases, a composition described herein may be used for treatment of shingles, chicken pox, measles, human papillomavirus (HPV), chronic obstructive pulmonary disease (COPD), emphysema, ilitigo, impetigo, pneumonia, listeria, Ebola, Addison's disease, Graves' disease, Sjögren's syndrome, Hashimoto's disease, autism, myasthenia gravis, Pernicious Anemia, or Celiac disease.

In some cases, a composition may be used to treat an autoimmune disease. In some cases, a composition may be used to treat Achalasia, Addison's disease, Adult Still's disease, Agammaglobulinemia, Alopecia arcata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner car discase (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenia purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue discase (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Parancoplastic cerebellar degeneration (PCD), Paroxysmal nocturnal hemoglobinuria (PNH), Parry Romberg syndrome, Pars planitis (peripheral uveitis), Parsonnage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenia purpura (TTP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, Vogt-Koyanagi-Harada Disease, or Wegener's granulomatosis (or Granulomatosis with Polyangiitis (GPA)).

A composition comprising cannabinoids may be formulated in water soluble form. Administration of the composition in a water soluble form may allow for rapid membrane absorption. The composition may be added to a water supply, e.g., drinking water, for protection after a chemical agent or toxicity event or exposure.

A composition may be used to enhance neurogenesis, or the growth and development of nervous tissue in a subject. A composition may also enhance the overall performance of the nervous system, including the parasympathetic nervous system, the sympathetic nervous system, and enteric nervous system.

A composition may be used as a supplement to protect a telomere, a region of the end of a chromosome in a subject. Protection of a telomere may protect the chromosome from deterioration.

In some cases, a composition described herein may be used as a targeted endocannabinoid system (ECB) therapeutic. Two primary endocannabinoid receptors of the endocannabinoid system are CB1 and CB2.

In some cases, a composition of the current disclosure may be used in combination with epigenetics, or the study of heritable changes in gene function that may not involve changes in the DNA sequence, or functionally relevant changes to the genome that may not involve a change in the nucleotide sequence (e.g. DNA methylation, histone modification). In some cases, epigenetic mechanisms may include factors and processes such as development (e.g. in utero, childhood), environmental factors (e.g. environmental chemicals), drugs, pharmaceuticals, aging, and dict.

A composition may be administered or suggested based on epigenetic testing. In some cases, a composition described herein can modulate risk factors or improve disease conditions. In some cases, terpenes, such as those described herein, may be used to direct cannabinoids to specific CB receptor sites. Compounds disclosed herein may be used to prevent to mitigate risks or harm during or after epigenetic indication and may contribute to changing the expression.

In some cases, a composition could be used to treat the thyroid if a thyroid risk factor was apparent, then the same composition could be used to target a different region of the body using different terpenes if a new epigenetic expression appeared. In some cases, a composition may have rapid absorption in the body. If a composition has rapid absorption, the same formula may be given sequentially and may change the effects of the cannabinoids.

In some cases, a composition may be administered or suggested based on genetic testing.

Alternatively, a composition may be administered based on standard testing for targeted treatment protocols, wherein cannabinoids and terpenes in the composition may prevent and/or treat risk factors or disease states.

A subject may exhibit one or more symptoms. A symptom may be selected from pain, stress, nausea, vomiting, sleeplessness, anxiety, and appetite loss. A unit dose may be used to alleviate a symptom in a subject, and in some cases, by at least about 10%, 20%, 30%, 40%, 50%, 60%, 70%, 75%, 80%, 85%, 90%, 95%, or 99%. A composition may result in a decrease of the severity or quantity of symptoms by at least about 10%, 20%, 30%, 40%, 50%, 60%, 70%, 75%, 80%, 85%, 90%, 95%, or 99%.

A unit dose can be administered at least about 1, 2, 3, 4, 5, 6, 7, 8, 9, 10 or more times a daily. A subject can receive dosing for a period of about, less than about, or greater than about 1, 2, 3, 4, 5, 6, 7, 8, 9, 10, 11, 12, 13, 14 or more days, weeks or months. A unit dose can be a fraction of the daily dose, such as the daily dose divided by the number of unit doses to be administered per day. A unit dose can be a fraction of the daily dose that is the daily dose divided by the number of unit doses to be administered per day and further divided by the number of unit doses (e.g. tablets) per administration. The number of unit doses per administration can be at least 1, 2, 3, 4, 5, 6, 7, 8, 9, 10, or more. The number of doses per day can at least 1, 2, 3, 4, 5, 6, 7, 8, 9, 10, or more. The number of unit doses per day can be determined by dividing the daily dose by the unit dose, and can at least 1, 2, 3, 4, 5, 6, 7, 8, 9, 10, 11, 12, 13, 14, 15, 6, 17, 18, 19, 20, or more unit doses per day. For example, a unit dose can be about ½, ⅓, ¼, ⅕, ⅙, 1/7, ⅛, 1/9, or 1/10 of the recommended daily dose. A unit dose can be about one-third of the daily amount and administered to the subject three times daily. A unit dose can be about one-half of the daily amount and administered to the subject twice daily. A unit dose can be about one-fourth of the daily amount with two unit doses administered to the subject twice daily.

The length of the period of administration and/or the dosing amounts can be determined by a physician or any other type of clinician. The physician or clinician can observe the subject's response to the administered compositions and adjust the dosing based on the subject's performance. For example, dosing for subjects that show reduced effects in energy regulation can be increased to achieve desired results.

The components in the compositions can be administered together at the same time in the same route, or administered separately. The components in the compositions can also be administered subsequently. The components in the compositions can be administered at the same or different administration route.

Another aspect of the present disclosure provides for achieving desired effects in one or more subjects after administration of a combination composition described herein for a specified time period. For example, the beneficial effects of the compositions described herein can be observed after administration of the compositions to the subject for 1, 2, 3, 4, 6, 8, 10, 12, 24, or 52 weeks.

In some embodiment, the present disclosure also provides for methods of manufacturing the compositions described herein. The manufacture of a composition described herein may comprise mixing or combining two or more components.

The compositions can be combined or mixed with a pharmaceutically active or therapeutic agent (i.e., a therapeutic compound), a carrier, and/or an excipient. Examples of such components are described herein. The combined compositions can be formed into a unit dosage as tablets, capsules, gel capsules, or slow-release tablets.

The composition may be prepared such that a solid composition containing a substantially homogeneous mixture of the one or more components is achieved, such that the one or more components are dispersed evenly throughout the composition so that the composition can be readily subdivided into equally effective unit dosage forms such as tablets, pills and capsules.

A unit dose of a composition may retain at least about 10%, 20%, 30%, 40%, 50%, 60%, 70%, 80%, 90%, or 95% of one or more cannabinoids after placement in a sealed container for a certain period of time, such as after 1 day, 7 days, 1 month, 6 months, 1 year, 2 years, 3 years, 4 years, 5 years, or more. A unit dose of a composition may have a shelf-life that is at least about 1 day, 7 days, 1 month, 6 months, 1 year, 2 years, 3 years, 4 years, 5 years, or more. Without wishing to be bound by theory, a trace amount of acid in a composition may contribute and enhance the shelf life of a composition.

A unit dose of a composition may be stored under conditions of at least about 25° C., 30° ° C., 40° C., 50° C., 60° C., 70° C., or more. A unit dose of a composition may be stored under a humidity level condition of at least about 10%, 20%, 30%, 40%, 50%, 60%, 70%, 80%, 90%, 95% or more humidity level without significant degradation of the cannabinoid.

A unit dose may be packaged into a container to be transferred to the user. A unit dose may be packaged in a tube, a jar, a box, a vial, a bag, a tray, a drum, a bottle, a syringe, or a can.

### Kits

The present disclosure also provides kits. The kits include one or more compositions described herein, in suitable packaging, and can further comprise written material that can include instructions for use, discussion of clinical studies, and listing of side effects. Such kits can also include information, such as scientific literature references, package insert materials, clinical trial results, and/or summaries of these, which indicate or establish the activities and/or advantages of the composition, and/or which describe dosing, administration, side effects, drug interactions, or other information useful to the health care provider. Such information can be based on the results of various studies, for example, studies using experimental animals involving in vivo models and studies based on human clinical trials. A kit can comprise one or more unit doses described herein. A kit may comprise at least about 1, 2, 3, 4, 5, 6, 7, 8, 9, 10, 11, 12, 13, 14, 15, 16, 17, 18, 19, 20, 30, 31, 60, 90, 120, 150, 180, 210, or more unit doses. A kit may comprise at most about 20, 15, 10, 5, 4, 3, 2, or 1 unit dose. Instructions for use can comprise dosing instructions, such as instructions to take 1, 2, 3, 4, 5, 6, 7, 8, 9, 10, or more unit doses 1, 2, 3, 4, 5, 6, 7, 8, 9, 10, or more times per day. For example, a kit can comprise a unit dose supplied as a tablet, with each tablet package separately, multiples of tablets packaged separately according to the number of unit doses per administration (e.g. pairs of tablets), or all tablets packaged together (e.g. in a bottle). As a further example, a kit can comprise a unit dose supplied as a bottled drink, the kit comprising 1, 2, 3, 4, 5, 6, 7, 8, 9, 10, 11, 12, 13, 14, 24, 28, 36, 48, 72, or more bottles.

Instructions may be provided in print form on a user interface of an electronic device of a user. The instructions may be provided, for example, on a graphical user interface or a web-based interface.

The kit can further contain another agent. The compound of the present disclosure and the agent may be provided or packaged as separate compositions in separate containers within the kit. The compound of the present disclosure and the agent may be provided or packaged as a single composition within a container in the kit. Suitable packaging and additional articles for use (e.g., measuring cup for liquid preparations, foil wrapping to minimize exposure to air) can be included in the kit. Kits described herein can be provided, marketed and/or promoted to health providers, including physicians, nurses, pharmacists, formulary officials. Kits can also be marketed directly to the consumer.

A kit can comprise a multi-day supply of unit dosages. The unit dosages can be any unit dosage described herein. The kit can comprise instructions directing the administration of the multi-day supply of unit dosages over a period of multiple days. The multi-day supply can be a one-month supply, a 30-day supply, or a multi-week supply. The multi-day supply can be a 90-day, 180-day, 3-month or 6-month supply. The kit can include packaged daily unit dosages, such as packages of 1, 2, 3, 4, or 5 unit dosages. The kit can be packaged with other dietary supplements, vitamins, and meal replacement bars, mixes, and beverages.

A kit may comprise starting materials that allows a user to perform the conversion of a carboxylated cannabinoid to a decarboxylated cannabinoid (e.g. 49 tetrahydrocannabinol).

A kit may comprise all the necessary starting materials so that a user may perform the conversion. The kit may comprise a carboxylated cannabinoid, an acid present to effect conversion of the carboxylated cannabinoid to a decarboxylated cannabinoid, a reaction vessel configured to hold a reaction mixture comprising the acid and the carboxylated cannabinoid, and instructions for performing the conversion utilizing the acid. The resulting decarboxylated cannabinoid that is formed from the conversion may be 49 tetrahydrocannabinol.

Alternatively, a kit may comprise some of the necessary starting materials so that a user may perform the conversion. A user may supplement the kit with his or her own supply of carboxylated cannabinoid. The kit may comprise an acid present to effect conversion of the carboxylated cannabinoid to a decarboxylated cannabinoid, a reaction vessel configured to hold a reaction mixture comprising the acid and the carboxylated cannabinoid, and instructions for performing the conversion utilizing the acid. The carboxylated cannabinoid that the user supplies may be tetrahydrocannabinolic acid.

The acid in a kit may be present in at least about 0.01 grams (g), 0.1 g, 0.5 g, 1 g, 2 g, 5 g, 10 g, 20 g, 30 g, 40 g, 50 g, 60 g, 70 g, 80 g, 90 g, 100 g, or more.

The carboxylated cannabinoid (e.g. tetrahydrocannabinolic acid), if provided in a kit, may be present in at least about 1 grams (g), 2 g, 5 g, 10 g, 20 g, 30 g, 40 g, 50 g, 60 g, 70 g, 80 g, 90 g, 100 g, or more. The amount of decarboxylated cannabinoid formed from performing a reaction using a kit may be at least about 1 grams (g), 2 g, 5 g, 10 g, 20 g, 30 g, 40 g, 50 g, 60 g, 70 g, 80 g, 90 g, 100 g, or more.

### EXAMPLES

### Example 1

A composition may comprise a terpene compound and a cannabinoid compound. The composition may comprise a cannabinoid hemp extract with cannabidiol (CBD) as the dominant cannabinoid, and the composition may further comprise one or more terpene compounds (e.g., one or more types of terpene compounds). A profile (e.g., a weigh ratio) of the cannabinoid compounds and the terpene compounds in such composition may be similar (e.g., substantially the same) as that of the Sour Diesel cannabis chemovar or an extract thereof. The terpene compound and the cannabinoid compound of the composition may be heterologous to each other, e.g., may be different sources. Alternatively, the terpene compound and the cannabinoid compound of the composition may be from the same source. In some cases, the terpene compound may be a non-cannabis terpene. In some cases, the terpene compound may be natural or synthetic. In some cases, the compositions disclosed herein may be encapsulated into single layer water soluble droplets. Such composition may, upon administration to a subject, induce an effect or experience to the subject that is similar to an effect or experience exerted on the user by consumption (e.g., via combustion or vaporization) of the Sour Diesel cannabis chemovar or the extract thereof.

### Example 2

A composition may be similar to that disclosed in Example #1, but with tetrahydrocannabinol (THC) as the predominant type of cannabinoid in the composition.

### Example 3

A composition may be similar to that disclosed in Example #1, but with a profile of the cannabinoid compounds and the terpene compounds in such composition being similar to that of OG Kush.

### Example 4

Another composition may be similar to that disclosed in Example #3, but with tetrahydrocannabinol (THC) as the predominant type of cannabinoid in the composition.

### Example 5

A composition may comprise terpenes and cannabinoids in a weight ratio that substantially matches that of a natural cannabis chemovar, such as Gorilla Glue (GG4). The weight ratio between one or more terpene compounds (e.g., all terpene compounds) and one or more cannabinoid compounds (e.g., all cannabinoid compounds) in the composition may be about 1:18. For example, the composition may comprise about 45 milligrams of terpene compounds per 1 gram of the composition. The composition may comprise about 820 grams of cannabinoid compounds per 1 gram of the composition.

The composition may comprise emulsion comprising a plurality of droplets (e.g., particles). The terpene compounds and the cannabinoid compounds may be encapsulated in a same droplet of the plurality of droplets, or in different droplets of the plurality of droplets.

### Example 6

Sour diesel's cannabinoid and/or terpene profile may have some variance (e.g., due to epigenetic drift). However, clone but true to type clones and tissue cultures will remain similar.

### Example 7

Compounds (e.g., terpene and/or cannabinoid compounds) disclosed herein can be extracted by a variety of methods. Extraction can be performed by, for example, maceration, infusion, decoction, percolation, Soxhlet extraction, pressurized solvent extraction, counter current extraction, ultrasonication, or supercritical fluid (e.g., carbon dioxide) extraction.

In some cases, one or more terpene compounds can be extracted (e.g., via carbon dioxide extraction) from a cannabis strain, and such extract can be combined with an additional extract from the cannabis strain (e.g., the same cannabis plant or a different cannabis plant but of the same strain), which additional extract comprising one or more cannabinoid compounds (e.g., decarboxylated or carboxylated cannabinoid compounds). For example, the resulting mixture composition may comprise at least one terpene compound and at least one cannabinoid compound at a first ratio (e.g., a weight ratio) that is substantially the same as a second ratio between the at least one terpene compound and at least one cannabinoid compound, which second ratio may be naturally present in the cannabis strain or a different cannabis strain.

In some cases, one or more terpene compounds can be extracted (e.g., via carbon dioxide extraction) from a cannabis strain, and such extract can be combined with (i) a different extract from a different cannabis strain, which different extract comprising one or more cannabinoid compounds, (ii) biosynthetically produced cannabinoids, or (iii) synthetic cannabinoids. For example, the resulting mixture composition may comprise at least one terpene compound and at least one cannabinoid compound at a first ratio (e.g., a weight ratio) that is substantially the same as a second ratio between the at least one terpene compound and at least one cannabinoid compound, which second ratio may be naturally present in the cannabis strain, the different cannabis strain, or yet another cannabis strain.

In some cases, one or more terpene compounds can be extracted (e.g., via carbon dioxide extraction) from a cannabis strain, and such extract can be combined with (i) a different extract from a different cannabis strain, which different extract comprising one or more cannabinoid compounds, (ii) biosynthetically produced cannabinoids, or (iii) synthetic cannabinoids. For example, the resulting mixture composition may comprise at least one terpene compound and at least one cannabinoid compound at a first ratio (e.g., a weight ratio) that is substantially the same as a second ratio between the at least one terpene compound and at least one cannabinoid compound, which second ratio may be naturally present in the cannabis strain, the different cannabis strain, or yet another cannabis strain.

### Example 8

Compounds (e.g., terpene and/or cannabinoid compounds) disclosed herein can be synthetically or biosynthetically produced. For example, a plurality of terpene compounds can be mixed at a specific ratio (e.g., a weight ratio) that substantially matches a ratio of the plurality of terpene compounds in a specific cannabis strain.

### Example 9

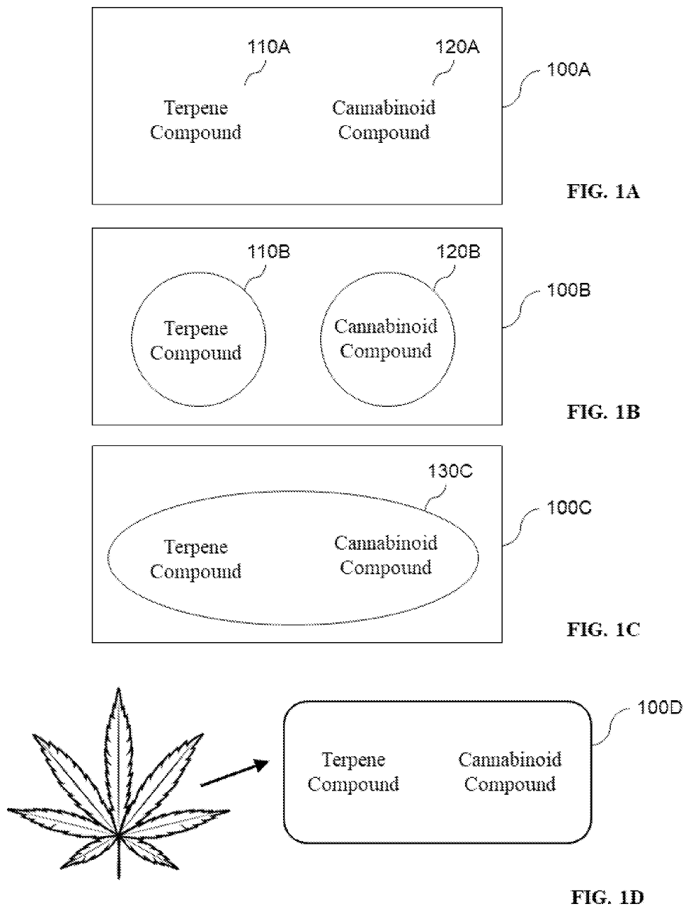

A therapeutic composition as disclosed herein can be administered to a subject having or is suspected of having a condition, such as PTSD or addiction. FIGS. 1A-1C show examples of therapeutic compositions comprising (i) at least one terpene compound and (ii) at least one cannabinoid compound, where each therapeutic composition is for being administered to a subject in need thereof. FIG. 1A shows an example composition 100A comprising at least one terpene compound that is non-encapsulated (110A) and at least one cannabinoid compound that is non-encapsulated (120A). FIG. 1B shows an example composition 100B comprising at least one terpene compound that is encapsulated in a first particle (110B) and at least one cannabinoid compound that is encapsulated in a different particle (120B). FIG. 1C shows an example composition 100C comprising at least one terpene compound and at least one cannabinoid compound that are encapsulated in the same particle (110C). The examples of FIGS. 1A-1C may not be a cannabis plant for an extract thereof. The examples of FIGS. 1A-IC may be, for example, manufactured compositions. In any of the examples in FIGS. 1A-IC, a weight ratio of the at least one terpene compound and the at least one cannabinoid compound may be substantially the same as a weight ratio of the at least one terpene compound and the at least one cannabinoid compound naturally present in a specific cannabis strain or an extract thereof, as represented as 100D in FIG. 1D.

### Example 10

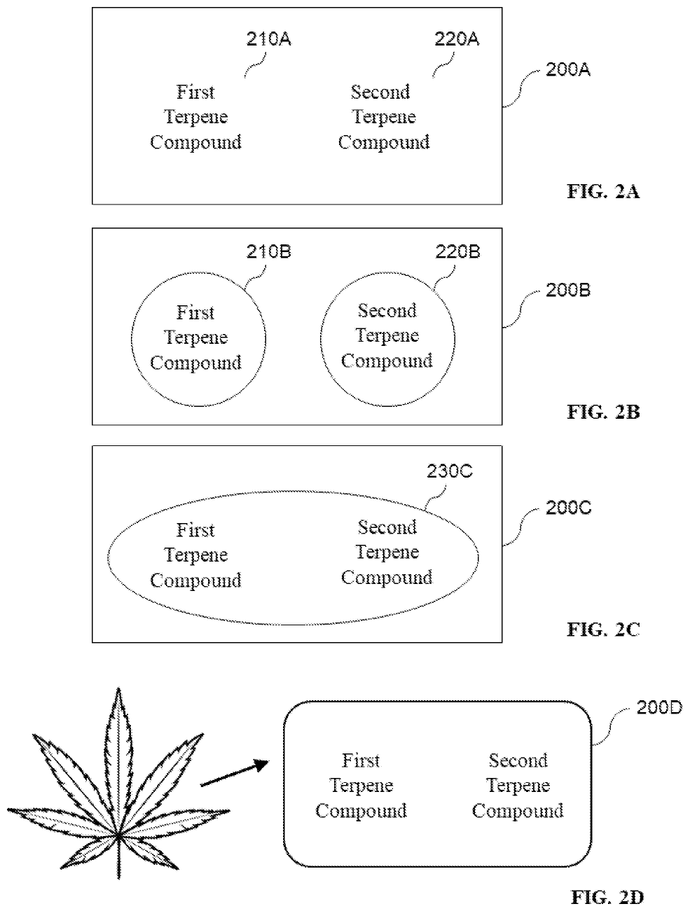

A therapeutic composition as disclosed herein can be administered to a subject having or is suspected of having a condition, such as PTSD or addiction. FIGS. 2A-2C show examples of therapeutic compositions comprising (i) a first terpene compound and (ii) a second terpene compound, where each therapeutic composition is for being administered to a subject in need thereof. FIG. 2A shows an example composition 200A comprising the first terpene compound that is non-encapsulated (210A) and the second terpene compound that is non-encapsulated (220A). FIG. 2B shows an example composition 200B comprising the first terpene compound that is encapsulated in a first particle (210B) and the second terpene compound that is encapsulated in a different particle (220B). FIG. 2C shows an example composition 200C comprising the first terpene compound and the second terpene compound that are encapsulated in the same particle (210C). The examples of FIGS. 2A-2C may not be a cannabis plant for an extract thereof. The examples of FIGS. 2A-2C may be, for example, manufactured compositions. In any of the examples in FIGS. 2A-2C, a weight ratio of the first terpene compound and the second terpene compound may be substantially the same as a weight ratio of the first terpene compound and the second terpene compound naturally present in a specific cannabis strain or an extract thereof, as represented as 200D in FIG. 2D. However, such composition may not comprise a full terpene spectrum of a specific cannabis strain or an extract thereof.

### Example 11

An internal standard (IS) mix is generated using methanol 4-vinyl-1-cyclohexene and D5-linalool at a concentration of 2 mg/ml. A cannabis sample of a Cannabis Strain 1 (CS1) is selected for terpene profile determination. The raw cannabis sample is transferred to a 20 ml headspace vial. The vial is sealed with a silicon rubber/teflon cap, placed in an autosampler, and incubated for 15 min at 160° C. The sample is then analyzed by a head space gas composition system. Terpenes in the terpene profile of the cannabis strain are identified by comparison with a series of standard solutions and/or matching mass spectra values with NIST library. The quantification of each terpene in the terpene profile is determined by comparing the extracted base peak areas for each analyte (e.g., terpene compound) against a calibration curve. The terpene compound concentrations of the terpenes present in the terpene profile of the CS1 are determined to be 340 ppm alpha-pinene, 10 ppm camphene, 295 ppm beta-pinene, 1070 ppm beta-mircene, 65 ppm 3-carene, 68 ppm alpha-terpinene, 10 ppm P-cimene, 362 ppm D-limonene, 7 ppm trans-beta-ocimene, 254 ppm cis-beta-ocimene, 57 ppm gamma-terpinene, 712 ppm terpinolene, 7 ppm L-fenchone, 138 ppm linalool, 191 ppm Fenchol, 9 ppm Borncol, 158 ppm DL-Menthol, 3 ppm beta-Citronellol, 4 ppm Geraniol, 23 ppm alpha-Cedrene, 1463 ppm beta-Caryophyllene, 384 ppm alpha-Humulene, 5 ppm trans-Nerolidol, 85 ppm beta-Eudesmol, 136 ppm alpha-Bisabolol. 10 ml of 1 mg/ml beta-Mircene stock solution is added to a reaction vessel. 14 ml of 1 mg/ml beta-Caryophyllene stock solution is added to the reaction vessel. 10 ml of 10 mg/ml CBD concentrate is added to the reaction vessel. 66 ml of oil is added to said reaction vessel. The beta-Caryophyllene, beta-Mircene and CBD are mixed in the reaction vessel to produce an emulsion with a weight ratio of beta-caryophyllene to beta-Mircene of 1:1.4—equivalent to the weight ratio of beta-Caryophyllene to beta-Mircene in the terpene profile of the sample of the cannabis strain.

### Further Embodiments

Embodiment 1: A composition comprising a terpene compound and a cannabinoid compound, wherein a weight ratio between said terpene compound and said cannabinoid compound in said composition (TC1) and a weight ratio between said terpene compound and said cannabinoid compound in a single cannabis strain (TC2) are different by no more than about 10%, wherein said composition is not a cannabis plant or a non-modified extract thereof.

Embodiment 2: The composition of embodiment 1, further comprising an emulsion, wherein said emulsion comprises said terpene compound and said cannabinoid compound.

Embodiment 3 The composition of embodiment 2, wherein said emulsion comprises a plurality of droplets, wherein a droplet of said plurality of droplets comprises said terpene compound and said cannabinoid compound.

Embodiment 4 The composition of embodiment 2, wherein said emulsion comprises a plurality of droplets, wherein said terpene compound and said cannabinoid compound are in different droplets of said plurality of droplets.

Embodiment 5: The composition of embodiment 1, wherein TC1 and TC2 are different by no more than about 10%.

Embodiment 6: The composition of embodiment 1, wherein TC1 and TC2 are different by no more than about 5%.

Embodiment 7: The composition of embodiment 1, wherein TC1 and TC2 are different by no more than about 1%.

Embodiment 8: The composition of embodiment 1, wherein TC1 and TC2 are substantially the same.

Embodiment 9: The composition of embodiment 1, wherein TC1 or TC2 is between about 50:1 and about 1:50.

Embodiment 10: The composition of embodiment 1, wherein TC1 or TC2 is between about 30:1 and about 1:30.

Embodiment 11: The composition of embodiment 1, wherein TC1 or TC2 is between about 1:10 and about 1:30.

Embodiment 12: The composition of embodiment 1, wherein said composition does not comprise a full-spectrum cannabinoid profile of said cannabis plant.

Embodiment 13: A composition comprising a plurality of terpene compounds comprising a terpene compound and an additional terpene compound that are different, wherein a weight ratio between said terpene compound and said additional terpene compound in said composition (TT1) and a weight ratio between said terpene compound and said additional terpene compound in a single cannabis strain (TT2) are different by no more than about 10%, and wherein said composition is not a cannabis plant or a non-modified extract thereof, and wherein said non-natural composition is substantially free of at least one type of terpene compound found in said cannabis plant.

Embodiment 14: The composition of embodiment 13, further comprising an emulsion, wherein said emulsion comprises said plurality of terpene compounds.

Embodiment 15: The composition of embodiment 14, wherein said emulsion comprises a plurality of droplets, wherein a droplet of said plurality of droplets comprises said terpene compound and said additional terpene compound.

Embodiment 16: The composition of embodiment 15, wherein said emulsion comprises a plurality of droplets, wherein said terpene compound and said additional terpene compound are in different droplets of said plurality of droplets.

Embodiment 17: The composition of embodiment 13, wherein TT1 and TT2 are different by no more than about 10%.

Embodiment 18: The composition of embodiment 13, wherein TT1 and TT2 are different by no more than about 5%.

Embodiment 19: The composition of embodiment 13, wherein TT1 and TT2 are different by no more than about 1%.

Embodiment 20: The composition of embodiment 13, wherein TT1 and TT2 are substantially the same.

Embodiment 21: The composition of embodiment 13, wherein TT1 or TT2 is between about 100:1 and about 1:100.

Embodiment 22: The composition of embodiment 13, wherein TT1 or TT2 is between about 50:1 and about 1:50.

Embodiment 23: The composition of embodiment 13, wherein TT1 or TT2 is between about 30:1 and about 1:30.

Embodiment 24: The composition of embodiment 13, wherein TT1 or TT2 is between about 10:1 and about 1:10.

Embodiment 25: The composition of embodiment 13, wherein said composition is substantially free of a plurality of types of terpene found in said cannabis plant.

Embodiment 26: The composition of embodiment 13, wherein said composition does not comprise a full-spectrum terpene profile of said cannabis plant.

Embodiment 27: The composition of embodiment 13, further comprising a cannabinoid compound.

Embodiment 28: The composition of any one of the preceding embodiments, wherein said single cannabis strain is selected from the group consisting of 9 Pound Hammer, Afghani, Afgoo, Berry White, Blueberry, Bubba Kush, G13, Granddaddy Purple, Grape Ape, Herijuana, Hindu Kush, Ingrid, Kosher Kush, Lavender, Master Kush, Northern Lights, Obama Kush, Pez, Plushberry, Presidential OG, Purple Urkle, Willy's Wonder, Zkittlez, Acdc, Ak-47, Banana OG, Blue Dream, Cannatonic, Chemdawg, Chernobyl, Cherry Pie, Cinderella 99, Dancehall, Double Dream, Dutch Treat, Ewok, Fruity Pebbles, Gelato, Golden Goat, Headband, Jeanguy, Jellybean, Juicy Fruit, Larry OG, Lemonder, Lodi Dodi, Mango Kush, Mendocino Purps, Middlefork, OG Kush, Permafrost, Pineapple Chunk, Pineapple Express, Pin Kush, Rascal OG, Sage, Sfv OG, Shiatsu Kush, Skunk No. 1, Snoop's Dream, Snowcap, Sour OG, Sour Tsunami, Space Queen, Sunset Sherbet, Tahoe OG, Tangerine Dream, Trainwreck, Uk Cheese, White Fire OG, White Widow, X3-X13, Acapulco Gold, Allen Wrench, Amnesia, Bay 11, Chocolope, Cinex, Dirty Girl, Durban Poison, Ghost Train Haze, Grapefuirt, Green Crack, Harlequin, Island Sweet Skunk, Jack Herer, Kali Mist, Lamb's Bread, Laghing Buddha, Maui Wowie, Panana Red, Purple Haze, Red Headed Stranger, Schrom, Sour Diesel, Strawberry Cough, Super Lemon Haze, Super Silver Haze, Tangie, Acapulco Gold, African, Cambodian red, Colombian, Hawaiian, Jamaican gold, Mexican red, Panama red, Thai stick, Amnesia Haze, Blueberry Diesel, Blue Goo, Bruce Banner, Bubblegum, Charlotte's Web, Critical Mass, Girl Scout Cookies, Gorilla Glue (GG4), Haze, Maui Waui, Platinum OG, Paonia Purple, Willie Nelson, Tochigishiro, and Feral cannabis.

Embodiment 29: The composition of any one of the preceding embodiments, wherein said terpene compound or said additional terpene compound is selected from the group consisting of myrcene, limonene, linalool, trans-ocimene, cis-ocimene, alpha-pinene, beta-pinene, alpha-humulene (alpha-caryophyllenc), beta-caryophyllene, delta-3-carene, trans-gamma-bisabolene, cis-gamma-bisabolene, trans-alpha-farnesene, cis-beta-farnesene, beta-fenchol, beta-phellandrene, guajol, alpha-gualene, alpha-cudesmol, beta-cudesmol, gamma-cudesmol, terpinolene, alpha-selinene, beta-selinene, alpha-terpineol, fenchone, camphene, cis-sabinene hydrate, alpha-trans-bergamotene, alpha-cis-bergamotene, borncol, gamma-curcumene, alpha-thujene, epi-alpha-bisabolol, ipsdienol, alpha-ylangene, beta-elemene, gamma-muurolene, alpha-cadinene, alpha-longipinene, and caryophyllene oxidc.

Embodiment 30: The composition of any one of the preceding embodiments, wherein said cannabinoid compound is selected from the group consisting of cannabigerol-type (CBG), cannabigerolic acid (CBGA), cannabigerolic acid monomethylether (CBGAM), cannabigerol monomethyl ether (CBGM), cannabichromene-type (CBC), cannabichromanon (CBCN), cannabichromenic acid (CBCA), cannabichromevarin-type (CBCV), cannabichromevarinic acid (CBCVA), cannabidiol-type (CBD), tetrahydrocannabinol-type (THC), iso-tetrahydrocannabinol-type (iso-THC), cannabinol-type (CBN), cannabinolic acid (CBNA), cannabinol methylether (CBNM), cannabinol-C₄ (CBN-C₄), cannabinol-C₂ (CBN-C₂), cannabiorcol (CBN-C₁), cannabinodiol (CBND), cannabielsoin-type (CBE), cannabielsoic acid A (CBEA-A), cannabielsoic acid B (CBEA-B), cannabicyclol-type (CBL), cannabicyclolic acid (CBLA), cannabicyclovarin (CBLV), cannabicitran-type (CBT), cannabitriol, cannabitriolvarin (CBTV), ethoxy-cannabitiolvarin (CBTVE), cannabivarin-type (CBV), cannabinodivarin (CBVD), tetrahydrocannabivarin-type (THCV), cannabidivarin-type (CBDV), cannabigerovarin-type (CBGV), cannabigerovarinic acid (CBGVA), cannabifuran (CBF), dehydrocannabifuran (DCBF), and cannabiripsol (CBR) cannabinoids.

Embodiment 31: The composition of any one of the preceding embodiments, wherein said cannabinoid compound comprises CBD.

Embodiment 32 The composition of any one of the preceding embodiments, wherein said cannabinoid compound comprises THC.

Embodiment 33: The composition of any one of the preceding embodiments, wherein said cannabinoid compound comprises a plurality of cannabinoid compounds.

Embodiment 34: The composition of any one of the preceding embodiments, further comprising a flavonoid.

Embodiment 35: A method for forming a composition comprising a terpene compound and a cannabinoid compound, the method comprising generating a mixture comprising said terpene compound and said cannabinoid compound, wherein a weight ratio between said terpene compound and said cannabinoid compound in said mixture and a weight ratio between said terpene compound and said cannabinoid compound in a single cannabis strain (TC2) are different by no more than about 10%, and wherein said composition is not a cannabis plant or a non-modified extract thereof.

Embodiment 36: A method for forming a composition comprising a plurality of terpene compounds comprising a terpene compound and an additional terpene compound that are different, the method comprising generating a mixture comprising said plurality of terpene compounds, wherein a weight ratio between said terpene compound and said additional terpene compound in said non-natural composition (TT1) and a weight ratio between said terpene compound and said additional terpene compound in a single cannabis strain (TT2) are different by no more than about 10%, wherein said non-natural composition is substantially free of at least one type of terpene compound found in said cannabis plant, and wherein said composition is not a cannabis plant or a non-modified extract thereof.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein can be employed in practicing the disclosure. It is intended that the following claims define the scope of the disclosure and that methods and structures within the scope of these claims and their equivalents be covered thereby.

1. A method of generating a cannabinoid formulation comprising:
(a) determining a terpene profile of a cannabis strain, wherein said terpene profile comprises at least a first terpene compound and a second terpene compound;
(b) providing a first amount of said first terpene compound, a second amount of said second terpene compound, and a third amount of a cannabinoid compound to a reaction vessel, wherein in said reaction vessel a weight ratio of said first terpene compound to said second terpene compound is substantially equivalent to a weight ratio of said first terpene compound to said second terpene compound in said terpene profile determined in (a);
(c) in said reaction vessel, mixing said first terpene, said second terpene, and said cannabinoid compound to generate an emulsion comprising said first terpene compound, said second terpene compound, and said cannabinoid compound.

2. The method of claim 1, wherein said emulsion comprises a plurality of droplets, wherein an individual droplet of said plurality of droplets has a size less than or equal to about 500 nanometers

3. The method of claim 2, wherein said droplet of said plurality of droplets comprises said first terpene compound or said second terpene compound.

4. The method of claim 2, wherein said droplet of said plurality of droplets comprises said first terpene compound, said second terpene compound, and said cannabinoid.

5. The method of claim 3 wherein said droplet is characterized by one or more members selected from the group consisting of: (i) a sigmoidal release profile of said first terpene compound, said second terpene compound, said cannabinoid, or a combination thereof, (ii) a plasma half-life of said first terpene compound, said second terpene compound, said cannabinoid, or a combination thereof greater than that of said first terpene compound, said second terpene compound, said cannabinoid, or said combination thereof in non-encapsulated form, (iii) a first pass metabolism of said first terpene compound, said second terpene compound, said cannabinoid, or a combination thereof, reduced by at least 5% compared to said first terpene compound, said second terpene compound, said cannabinoid, or said combination thereof, in non-encapsulated form, (iv) a rate of excretion of said first terpene compound, said second terpene compound, said cannabinoid, or a combination thereof, from a subject's body reduced by at least 5% compared to said first terpene compound, said second terpene compound, said cannabinoid, or said combination thereof in non-encapsulated form, and (v) a degradation rate at an ambient temperature of at least 20º Celsius (° C.) of said first terpene compound, said second terpene compound, said cannabinoid, or a combination thereof, of at least 5% less than a degradation rate of said first terpene compound, said second terpene compound, said cannabinoid, or said combination thereof, in non-encapsulated form.

6. The method of claim 1, wherein said first terpene compound and said second terpene compound are provided to said reaction vessel sequentially before said cannabinoid.

7. (canceled)

8. The method of claim 1, wherein said first amount of said first terpene compound is less than 5000 parts per million of said emulsion.

9. The method of claim 8, wherein said second amount of said second compound is less than said first amount of said first terpene compound.

10. The method of claim 1, wherein said emulsion is water soluble.

11. The method of claim 1, wherein a content of said cannabinoid compound, said first terpene compound or said second terpene compound is at least about 0.1% by weight of said droplet.

12. The method of claim 1, wherein said first terpene compound and said second terpene compound are in different droplets of said plurality of droplets.

13. The method of claim 1, wherein said terpene profile is determined, at least in part, by mass spectrometry of said cannabis strain.

14. The method of claim 1, wherein said emulsion comprises only a portion of said terpene compounds in said terpene profile.

15-17. (canceled)

18. The method of claim 1, wherein said cannabinoid comprises CBD or THC.

20. The method of claim 1, wherein said emulsion is substantially free of at least one additional terpene compound that is present in said terpene profile determined in (a).

21. (canceled)

22. A composition comprising a terpene compound and a cannabinoid compound, wherein the weight ratio of said terpene compound and said cannabinoid compound (TC1) is different by no more than about 10% as compared to the weight ratio between said terpene compound and said cannabinoid compound in a single cannabis strain (TC2), wherein said composition is not a cannabis plant or a non-modified extract thereof; and
said composition is an emulsion.

23. The composition according to claim 22, wherein the terpene compound and cannabinoid are contained in droplets of the emulsion.

24. A composition comprising a first terpene compound and a second terpene compound, wherein the weight ratio of said first terpene compound and said second terpene compound (TT1) is different by no more than about 10% as compared to the weight ratio between said first terpene compound and said second terpene compound in a single cannabis strain (TT2),
wherein said composition is substantially free of at least one type of terpene compound found in said cannabis strain; and
said composition is in the form of an emulsion.

25. The composition according to claim 24, wherein said first terpene compound and said second terpene compound are contained in droplets of the emulsion.
